SEQUENCE LISTING (437 sequences)

SEQ ID NO: 1 - DNA, 20 bp, homo sapiens
tctttggcattaggagcttg

SEQ ID NO: 2 - DNA, 20 bp, homo sapiens
caaacccaacccatacacac

SEQ ID NO: 3 - DNA, 25 bp, homo sapiens
gttgtaaatcctggacctgcagaag

SEQ ID NO: 4 - DNA, 24 bp, homo sapiens
ccctcctctgattccacaaggtat

SEQ ID NO: 5 - DNA, 20 bp, homo sapiens
tccatatgccagaaaagttg

SEQ ID NO: 6 - DNA, 20 bp, homo sapiens
acaagcatgcactaccattc

SEQ ID NO: 7 - DNA, 21 bp, homo sapiens
cttgggttaatctccttggat

SEQ ID NO: 8 - DNA, 20 bp, homo sapiens
cacctattcaccagatttcg

SEQ ID NO: 9 - DNA, 24 bp, homo sapiens
ccttagcaatttgtatgagcccaa

SEQ ID NO: 10 - DNA, 26 bp, homo sapiens
ccatcataggatacaatgaatgctgg

SEQ ID NO: 11 - DNA, 25 bp, homo sapiens
ccactgttgctataacaaatcccaa

SEQ ID NO: 12 - DNA, 26 bp, homo sapiens
ttcagcatttatcccttacttgtacc

SEQ ID NO: 13 - DNA, 20 bp, homo sapiens
atttctgcctagatgctggg

SEQ ID NO: 14 - DNA, 20 bp, homo sapiens
aactccgcctttccagttgt

SEQ ID NO: 15 - DNA, 20 bp, homo sapiens
gctcagaaccacgaagtgtt

SEQ ID NO: 16 - DNA, 20 bp, homo sapiens
catcatcattctccctagcc

SEQ ID NO: 17 - DNA, 24 bp, homo sapiens
ggcacataggaggcatttaccaaa

SEQ ID NO: 18 - DNA, 26 bp, homo sapiens
gtcacaaacatcaaatatgaggtgga

SEQ ID NO: 19 - DNA, 24 bp, homo sapiens
gaccatgctcagatcttccattcc

SEQ ID NO: 20 - DNA, 25 bp, homo sapiens
ccactctcatccatcatactgtcca

SEQ ID NO: 21 - DNA, 24 bp, homo sapiens
agtgggtaattcagggttgctttg

SEQ ID NO: 22 - DNA, 24 bp, homo sapiens
cacctggccattcctctacttctt

SEQ ID NO: 23 - DNA, 19 bp, homo sapiens
ggccatgtgcttttcaaac

SEQ ID NO: 24 - DNA, 22 bp, homo sapiens
ctccaaaaataccttccagcac

SEQ ID NO: 25 - DNA, 24 bp, homo sapiens
tgtgcccttctctgtgaacctcta

SEQ ID NO: 26 - DNA, 25 bp, homo sapiens
tgatccattcacagtagcttaccca

SEQ ID NO: 27 - DNA, 22 bp, homo sapiens
tgtggttaaagcaatagtgtga

SEQ ID NO: 28 - DNA, 20 bp, homo sapiens
acagcaaatgcttgctagac

SEQ ID NO: 29 - DNA, 23 bp, homo sapiens
tgtagctcttgcatactgtcttc

SEQ ID NO: 30 - DNA, 21 bp, homo sapiens
agtgctgccacaactgtataa

SEQ ID NO: 31 - DNA, 25 bp, homo sapiens
cttcagcagttcttggaatgttgtg

SEQ ID NO: 32 - DNA, 24 bp, homo sapiens
ggaagtaatcttgaatcctggccc

SEQ ID NO: 33 - DNA, 24 bp, homo sapiens
atgctaaaatacgagacatattgc

SEQ ID NO: 34 - DNA, 25 bp, homo sapiens
acatgctacatattgcattctactc

SEQ ID NO: 35 - DNA, 24 bp, homo sapiens
cattattctggctatagaatgaca

SEQ ID NO: 36 - DNA, 24 bp, homo sapiens
tgtatacatccccaaactatctta

SEQ ID NO: 37 - DNA, 20 bp, homo sapiens
gcatgggaggaataggtgaa

SEQ ID NO: 38 - DNA, 20 bp, homo sapiens
tgcttgggagaaatgaaaca

SEQ ID NO: 39 - DNA, 24 bp, homo sapiens
gtgcagctcctgcagtttctaaag

SEQ ID NO: 40 - DNA, 22 bp, homo sapiens
accaacaaaaccacaggcccta

SEQ ID NO: 41 - DNA, 25 bp, homo sapiens
gcaggatgagtacccacctattcct

SEQ ID NO: 42 - DNA, 24 bp, homo sapiens
gaaagatggtcctttgtgcctctc

SEQ ID NO: 43 - DNA, 25 bp, homo sapiens
cactgacacactttgtccactttgc

SEQ ID NO: 44 - DNA, 24 bp, homo sapiens
gaatgtcctgtacaccaactgtgg

SEQ ID NO: 45 - DNA, 24 bp, homo sapiens
tattcaaagaatggcaccagtgtg

SEQ ID NO: 46 - DNA, 23 bp, homo sapiens
gacaatctgtgtgcatcggtttt

SEQ ID NO: 47 - DNA, 20 bp, homo sapiens
gtgccctaggagaagtgtga

SEQ ID NO: 48 - DNA, 22 bp, homo sapiens
gacagatacacagtgaccctca

SEQ ID NO: 49 - DNA, 24 bp, homo sapiens
cctgttagttcattgaaaagcccg

SEQ ID NO: 50 - DNA, 24 bp, homo sapiens
ttctgctaacacattgcttcaggc

SEQ ID NO: 51 - DNA, 20 bp, homo sapiens
gatttctggagaccacaagg

SEQ ID NO: 52 - DNA, 20 bp, homo sapiens
acctcctccctgagaatgtt

SEQ ID NO: 53 - DNA, 20 bp, homo sapiens
atcttccactggtgacagga

SEQ ID NO: 54 - DNA, 21 bp, homo sapiens
ctgcctatgagaaaactgcac

SEQ ID NO: 55 - DNA, 20 bp, homo sapiens
aatgttcacaagggactcca

SEQ ID NO: 56 - DNA, 20 bp, homo sapiens
cctgttgctccaggtatgtt

SEQ ID NO: 57 - DNA, 24 bp, homo sapiens
tgccttctgtcccagatctcacta

SEQ ID NO: 58 - DNA, 24 bp, homo sapiens
tcagtgtcctcaattccccttacc

SEQ ID NO: 59 - DNA, 36 bp, homo sapiens
gaacaattaactcaaatatacacaaggcttgtcttt

SEQ ID NO: 60 - DNA, 36 bp, homo sapiens
caaaagtttaagattttgcttttggcagggtaaaag

SEQ ID NO: 61 - DNA, 25 bp, homo sapiens
ggaaaaggccagcgttgtctccaaa

SEQ ID NO: 62 - DNA, 25 bp, homo sapiens
tggccaccttctcacctgaaaaaaa

SEQ ID NO: 63 - DNA, 25 bp, homo sapiens
gttcctcctctctttattttagctg

SEQ ID NO: 64 - DNA, 25 bp, homo sapiens
atcctttcctcaaaattggtctggt

SEQ ID NO: 65 - DNA, 25 bp, homo sapiens
gcgcctggaattgtcagacatatac

SEQ ID NO: 66 - DNA, 25 bp, homo sapiens
tcagcagaatcaacagaagggattt

SEQ ID NO: 67 - DNA, 25 bp, homo sapiens
gaaaaattggaaaggtatgttcatg

SEQ ID NO: 68 - DNA, 25 bp, homo sapiens
atttctctcttcaactaaacaatgt

SEQ ID NO: 69 - DNA, 25 bp, homo sapiens
cactttttattcttttgcagagaat

SEQ ID NO: 70 - DNA, 25 bp, homo sapiens
tttctttgaagccagctctctatcc

SEQ ID NO: 71 - DNA, 25 bp, homo sapiens
actttttattcttttgcagagaatg

SEQ ID NO: 72 - DNA, 25 bp, homo sapiens
ttttctttgaagccagctctctatc

SEQ ID NO: 73 - DNA, 25 bp, homo sapiens
attcttttgcagagaatgggataga

SEQ ID NO: 74 - DNA, 25 bp, homo sapiens
ttaggatttttctttgaagccagct

SEQ ID NO: 75 - DNA, 25 bp, homo sapiens
agagagctggcttcaaagaaaaatc

SEQ ID NO: 76 - DNA, 25 bp, homo sapiens
tcgccgaagggcattaatgagttta

SEQ ID NO: 77 - DNA, 25 bp, homo sapiens
aatcctaaactcattaatgcccttc

SEQ ID NO: 78 - DNA, 25 bp, homo sapiens
cataaatctccagaaaaaacatcgc

SEQ ID NO: 79 - DNA, 25 bp, homo sapiens
tcctaaactcattaatgcccttcgg

SEQ ID NO: 80 - DNA, 25 bp, homo sapiens
aacataaatctccagaaaaaacatc

SEQ ID NO: 81 - DNA, 25 bp, homo sapiens
ttaatgcccttcggcgatgtttttt

SEQ ID NO: 82 - DNA, 25 bp, homo sapiens
atagaacataaatctccagaaaaaa

SEQ ID NO: 83 - DNA, 25 bp, homo sapiens
tttttctggagatttatgttctatg

SEQ ID NO: 84 - DNA, 25 bp, homo sapiens
ccttacccctaaatataaaaagatt

SEQ ID NO: 85 - DNA, 25 bp, homo sapiens
gagatttatgttctatggaatcttt

SEQ ID NO: 86 - DNA, 25 bp, homo sapiens
tgagatccttacccctaaatataaa

SEQ ID NO: 87 - DNA, 25 bp, homo sapiens
gaatgtacaaatgagatccttaccc

SEQ ID NO: 88 - DNA, 25 bp, homo sapiens
gttctatggaatctttttatattta

SEQ ID NO: 89 - DNA, 25 bp, homo sapiens
ctatggaatctttttatatttaggg

SEQ ID NO: 90 - DNA, 25 bp, homo sapiens
aatgaatgtacaaatgagatcctta

SEQ ID NO: 91 - DNA, 25 bp, homo sapiens
atggaatctttttatatttaggggt

SEQ ID NO: 92 - DNA, 25 bp, homo sapiens
ataatgaatgtacaaatgagatcct

SEQ ID NO: 93 - DNA, 25 bp, homo sapiens
atttctctgtttttccccttttgta

SEQ ID NO: 94 - DNA, 25 bp, homo sapiens
gaggctgtactgctttggtgacttc

SEQ ID NO: 95 - DNA, 25 bp, homo sapiens
tttctctgtttttccccttttgtag

SEQ ID NO: 96 - DNA, 25 bp, homo sapiens
agaggctgtactgctttggtgactt

SEQ ID NO: 97 - DNA, 25 bp, homo sapiens
ttgtaggaagtcaccaaagcagtac

SEQ ID NO: 98 - DNA, 25 bp, homo sapiens
tatgattcttcccagtaagagaggc

SEQ ID NO: 99 - DNA, 25 bp, homo sapiens
agtacagcctctcttactgggaaga

SEQ ID NO: 100 - DNA, 25 bp, homo sapiens
tatccgggtcataggaagctatgat

SEQ ID NO: 101 - DNA, 25 bp, homo sapiens
cttactgggaagaatcatagcttcc

SEQ ID NO: 102 - DNA, 25 bp, homo sapiens
agcgttcctccttgttatccgggtc

SEQ ID NO: 103 - DNA, 25 bp, homo sapiens
ctatgacccggataacaaggaggaa

SEQ ID NO: 104 - DNA, 25 bp, homo sapiens
atgcctagataaatcgcgatagagc

SEQ ID NO: 105 - DNA, 24 bp, homo sapiens
atgacccggataacaaggaggaac

SEQ ID NO: 106 - DNA, 25 bp, homo sapiens
tatgcctagataaatcgcgatagag

SEQ ID NO: 107 - DNA, 25 bp, homo sapiens
aggaggaacgctctatcgcgattta

SEQ ID NO: 108 - DNA, 31 bp, homo sapiens
gaataaagagaagacataagcctatgcctag

SEQ ID NO: 109 - DNA, 25 bp, homo sapiens
cccagccatttttggccttcatcac

SEQ ID NO: 110 - DNA, 25 bp, homo sapiens
atagctattctcatctgcattccaa

SEQ ID NO: 111 - DNA, 25 bp, homo sapiens
ccagccatttttggccttcatcaca

SEQ ID NO: 112 - DNA, 25 bp, homo sapiens
catagctattctcatctgcattcca

SEQ ID NO: 113 - DNA, 25 bp, homo sapiens
ctatgtttagtttgatttataagaa

SEQ ID NO: 114 - DNA, 25 bp, homo sapiens
tggggcctgtgcaaggaagtattac

SEQ ID NO: 115 - DNA, 25 bp, homo sapiens
tatgtttagtttgatttataagaag

SEQ ID NO: 116 - DNA, 25 bp, homo sapiens
atggggcctgtgcaaggaagtatta

SEQ ID NO: 117 - DNA, 25 bp, homo sapiens
aactttccatttttcttttagactt

SEQ ID NO: 118 - DNA, 25 bp, homo sapiens
ctagaacacggcttgacagctttaa

SEQ ID NO: 119 - DNA, 25 bp, homo sapiens
gccgtgttctagataaaataagtat

SEQ ID NO: 120 - DNA, 25 bp, homo sapiens
ggaaaggagactaacaagttgtcca

SEQ ID NO: 121 - DNA, 25 bp, homo sapiens
ccgtgttctagataaaataagtatt

SEQ ID NO: 122 - DNA, 25 bp, homo sapiens
ttggaaaggagactaacaagttgtc

SEQ ID NO: 123 - DNA, 25 bp, homo sapiens
tgttctagataaaataagtattgga

SEQ ID NO: 124 - DNA, 25 bp, homo sapiens
ttgttggaaaggagactaacaagtt

SEQ ID NO: 125 - DNA, 25 bp, homo sapiens
caacaacctgaacaaatttgatgaa

SEQ ID NO: 126 - DNA, 25 bp, homo sapiens
aaaagattaaatcaataggtacata

SEQ ID NO: 127 - DNA, 24 bp, homo sapiens
acctgaacaaatttgatgaagtat

SEQ ID NO: 128 - DNA, 25 bp, homo sapiens
gcctaaaagattaaatcaataggta

SEQ ID NO: 129 - DNA, 25 bp, homo sapiens
gtttttgctgtgcttttattttcca

SEQ ID NO: 130 - DNA, 25 bp, homo sapiens
acacgaaatgtgccaatgcaagtcc

SEQ ID NO: 131 - DNA, 25 bp, homo sapiens
attggcacatttcgtgtggatcgct

SEQ ID NO: 132 - DNA, 25 bp, homo sapiens
cccatgaggagtgccacttgcaaag

SEQ ID NO: 133 - DNA, 25 bp, homo sapiens
gcacatttcgtgtggatcgctcctt

SEQ ID NO: 134 - DNA, 25 bp, homo sapiens
tagccccatgaggagtgccacttgc

SEQ ID NO: 135 - DNA, 25 bp, homo sapiens
tattttccagggactagcattggca

SEQ ID NO: 136 - DNA, 25 bp, homo sapiens
tgcaaaggagcgatccacacgaaat

SEQ ID NO: 137 - DNA, 25 bp, homo sapiens
catggggctaatctgggagttgtta

SEQ ID NO: 138 - DNA, 25 bp, homo sapiens
ccaagtccacagaaagcagacgcct

SEQ ID NO: 139 - DNA, 25 bp, homo sapiens
gattacctcagaaatgattgaaaat

SEQ ID NO: 140 - DNA, 25 bp, homo sapiens
agcagtatgccttaacagattggat

SEQ ID NO: 141 - DNA, 25 bp, homo sapiens
gtgattacctcagaaatgattgaaa

SEQ ID NO: 142 - DNA, 25 bp, homo sapiens
gtatgccttaacagattggatattt

SEQ ID NO: 143 - DNA, 25 bp, homo sapiens
agaagcaatggaaaaaatgattgaa

SEQ ID NO: 144 - DNA, 25 bp, homo sapiens
attggaacaacttactgtcttaagt

SEQ ID NO: 145 - DNA, 25 bp, homo sapiens
tgaattttattgttattgtttttta

SEQ ID NO: 146 - DNA, 25 bp, homo sapiens
tccgagtcagtttcagttctgttct

SEQ ID NO: 147 - DNA, 27 bp, homo sapiens
gatacttcaatagctcagccttcttct

SEQ ID NO: 148 - DNA, 25 bp, homo sapiens
caccacaaagaaccctgagaagaag

SEQ ID NO: 149 - DNA, 25 bp, homo sapiens
cagccttcttcttctcagggttctt

SEQ ID NO: 150 - DNA, 25 bp, homo sapiens
ggaagcacagataaaaacaccacaa

SEQ ID NO: 151 - DNA, 25 bp, homo sapiens
ctgtgcttccctatgcactaatcaa

SEQ ID NO: 152 - DNA, 25 bp, homo sapiens
aatattttccggaggatgattcctt

SEQ ID NO: 153 - DNA, 23 bp, homo sapiens
tgcttccctatgcactaatcaaa

SEQ ID NO: 154 - DNA, 25 bp, homo sapiens
gtgaatattttccggaggatgattc

SEQ ID NO: 155 - DNA, 25 bp, homo sapiens
tgcactaatcaaaggaatcatcctc

SEQ ID NO: 156 - DNA, 25 bp, homo sapiens
aatgagatggtggtgaatattttcc

SEQ ID NO: 157 - DNA, 25 bp, homo sapiens
atcaaaggaatcatcctccggaaaa

SEQ ID NO: 158 - DNA, 25 bp, homo sapiens
aatacagaatgagatggtggtgaat

SEQ ID NO: 159 - DNA, 25 bp, homo sapiens
ggaatcatcctccggaaaatattca

SEQ ID NO: 160 - DNA, 25 bp, homo sapiens
cagaacaatgcagaatgagatggtg

SEQ ID NO: 161 - DNA, 25 bp, homo sapiens
tccggaaaatattcaccaccatctc

SEQ ID NO: 162 - DNA, 25 bp, homo sapiens
atgcgcagaacaatgcagaatgaga

SEQ ID NO: 163 - DNA, 25 bp, homo sapiens
aaatattcaccaccatctcattctg

SEQ ID NO: 164 - DNA, 25 bp, homo sapiens
gagtgaccgccatgcgcagaacaat

SEQ ID NO: 165 - DNA, 25 bp, homo sapiens
accaccatctcattctgcattgttc

SEQ ID NO: 166 - DNA, 25 bp, homo sapiens
aaattgccgagtgaccgccatgcgc

SEQ ID NO: 167 - DNA, 25 bp, homo sapiens
accatctcattctacattgttctgc

SEQ ID NO: 168 - DNA, 25 bp, homo sapiens
gggaaattgccgagtgaccgccatg

SEQ ID NO: 169 - DNA, 25 bp, homo sapiens
attgttctgcgcatggcggtcactc

SEQ ID NO: 170 - DNA, 25 bp, homo sapiens
tgtttgtacagcccagggaaattgc

SEQ ID NO: 171 - DNA, 25 bp, homo sapiens
cactcggcaatttccctgggctgta

SEQ ID NO: 172 - DNA, 25 bp, homo sapiens
gctccaagagagtcataccatgttt

SEQ ID NO: 173 - DNA, 25 bp, homo sapiens
cggcaatttccctgggctatacaaa

SEQ ID NO: 174 - DNA, 25 bp, homo sapiens
cggcaatttccctgggctgtaaaaa

SEQ ID NO: 175 - DNA, 25 bp, homo sapiens
gctccaagagagtcataccattttt

SEQ ID NO: 176 - DNA, 25 bp, homo sapiens
tattgctccaagagagtcataccat

SEQ ID NO: 177 - DNA, 25 bp, homo sapiens
ctgggctgtacaaacatggtatgac

SEQ ID NO: 178 - DNA, 25 bp, homo sapiens
tgtattttgtttattgctccaagag

SEQ ID NO: 179 - DNA, 25 bp, homo sapiens
tctataaataggatttcttacaaaa

SEQ ID NO: 180 - DNA, 25 bp, homo sapiens
tccaatgtcttatattcttgctttt

SEQ ID NO: 181 - DNA, 25 bp, homo sapiens
gtgatggagaatgtaacagccttct

SEQ ID NO: 182 - DNA, 25 bp, homo sapiens
ttttttaaaaattctgacctcctcc

SEQ ID NO: 183 - DNA, 25 bp, homo sapiens
tgatggagaatgtaacagccttctg

SEQ ID NO: 184 - DNA, 25 bp, homo sapiens
attttttaaaaattctgacctcctc

SEQ ID NO: 185 - DNA, 25 bp, homo sapiens
tgtgtgtgtgtgtgtgtgtgttttt

SEQ ID NO: 186 - DNA, 23 bp, homo sapiens
attccccaaatccctgttaaaaa

SEQ ID NO: 187 - DNA, 25 bp, homo sapiens
tgtgtgtgtgtgtgtgtgttttttt

SEQ ID NO: 188 - DNA, 25 bp, homo sapiens
attccccaaatccctgttaaaaaaa

SEQ ID NO: 189 - DNA, 27 bp, homo sapiens
attccccaaatccctgttaaaaaaaaa

SEQ ID NO: 190 - DNA, 25 bp, homo sapiens
attccccaaatccctgttaaaaaca

SEQ ID NO: 191 - DNA, 25 bp, homo sapiens
gtgtgtgtgtgtgtgtttttttaac

SEQ ID NO: 192 - DNA, 25 bp, homo sapiens
tttctcaaataattccccaaatccc

SEQ ID NO: 193 - DNA, 25 bp, homo sapiens
aagatagaaagaggacagttgttgg

SEQ ID NO: 194 - DNA, 25 bp, homo sapiens
gcctgctccagtggatccagcaacc

SEQ ID NO: 195 - DNA, 25 bp, homo sapiens
agaggacagttgttggcggttgctg

SEQ ID NO: 196 - DNA, 25 bp, homo sapiens
actaccttgcctgctacagtggatc

SEQ ID NO: 197 - DNA, 25 bp, homo sapiens
tatgggagaactggagccttcagag

SEQ ID NO: 198 - DNA, 25 bp, homo sapiens
attcttccactgtgcttaattttac

SEQ ID NO: 199 - DNA, 28 bp, homo sapiens
gcacagtggaagactttcattctgttct

SEQ ID NO: 200 - DNA, 25 bp, homo sapiens
gtgacaggcataatccaggaaaact

SEQ ID NO: 201 - DNA, 25 bp, homo sapiens
cagtggaagaatttcattctgttct

SEQ ID NO: 202 - DNA, 25 bp, homo sapiens
ggtgccaggcataatccaggaaaac

SEQ ID NO: 203 - DNA, 25 bp, homo sapiens
gcctggcaccattaaagaaaatatc

SEQ ID NO: 204 - DNA, 25 bp, homo sapiens
attcatcataggaaacaccaaagat

SEQ ID NO: 205 - DNA, 28 bp, homo sapiens
agcctggcaccattaaagaaaatatcat

SEQ ID NO: 206 - DNA, 25 bp, homo sapiens
ctatattcatcataggaaacaccaa

SEQ ID NO: 207 - DNA, 25 bp, homo sapiens
tctttggtgtttcctatgatgaata

SEQ ID NO: 208 - DNA, 25 bp, homo sapiens
tgatgacgcttctgtatctatattc

SEQ ID NO: 209 - DNA, 25 bp, homo sapiens
tttgatgacgcttctgtatctattc

SEQ ID NO: 210 - DNA, 25 bp, homo sapiens
ctatgatgaatatagatacagaagc

SEQ ID NO: 211 - DNA, 25 bp, homo sapiens
tcttctagttggcttgctttgatga

SEQ ID NO: 212 - DNA, 25 bp, homo sapiens
gatacagaagcgtcatcaaagcatg

SEQ ID NO: 213 - DNA, 25 bp, homo sapiens
catagtttcttacctcttctagttg

SEQ ID NO: 214 - DNA, 25 bp, homo sapiens
agtgactctctaattttctattttt

SEQ ID NO: 215 - DNA, 25 bp, homo sapiens
tgcaaacttggagatgtcctattac

SEQ ID NO: 216 - DNA, 25 bp, homo sapiens
ctctaattttctatttttggtaata

SEQ ID NO: 217 - DNA, 25 bp, homo sapiens
ctttctctgcaaacttggagatgtc

SEQ ID NO: 218 - DNA, 25 bp, homo sapiens
tgcagagaaagacaatatagttctt

SEQ ID NO: 219 - DNA, 25 bp, homo sapiens
ccactcagtgtgattccaccttctc

SEQ ID NO: 220 - DNA, 25 bp, homo sapiens
tcttggagaaggtagaatcacactg

SEQ ID NO: 221 - DNA, 25 bp, homo sapiens
gaaattcttgctcgttgacctccac

SEQ ID NO: 222 - DNA, 25 bp, homo sapiens
cttggagaaggtggaatcacactga

SEQ ID NO: 223 - DNA, 25 bp, homo sapiens
tgaaattcttgctcgttgacctcca

SEQ ID NO: 224 - DNA, 25 bp, homo sapiens
ttggagaaggtggaatcacactgag

SEQ ID NO: 225 - DNA, 25 bp, homo sapiens
aagaaatacttgctcgttgacctcc

SEQ ID NO: 226 - DNA, 25 bp, homo sapiens
gaaggtggaatcacactgagtggag

SEQ ID NO: 227 - DNA, 25 bp, homo sapiens
tgctaaagaaattcttgctcgttga

SEQ ID NO: 228 - DNA, 25 bp, homo sapiens
gaaggtggaatcacactgagtggag

SEQ ID NO: 229 - DNA, 25 bp, homo sapiens
gctaaagaaattcttgctcgttgac

SEQ ID NO: 230 - DNA, 25 bp, homo sapiens
aggtggaatcacactgagtggaggt

SEQ ID NO: 231 - DNA, 25 bp, homo sapiens
cttgctaaagaaattcttgctcgtt

SEQ ID NO: 232 - DNA, 25 bp, homo sapiens
tggaatcacactgagtggaggtcaa

SEQ ID NO: 233 - DNA, 25 bp, homo sapiens
caccttgctaaagaaattcttgctc

SEQ ID NO: 234 - DNA, 25 bp, homo sapiens
ggaatcacactgagtggaggtcaac

SEQ ID NO: 235 - DNA, 25 bp, homo sapiens
tcaccttgctaaagaaattcttgct

SEQ ID NO: 236 - DNA, 25 bp, homo sapiens
ggaggtcaacgagcgagaatttctt

SEQ ID NO: 237 - DNA, 25 bp, homo sapiens
ccaataattagttattcaccttgct

SEQ ID NO: 238 - DNA, 25 bp, homo sapiens
aggtcaacgagcaagaatttcttta

SEQ ID NO: 239 - DNA, 25 bp, homo sapiens
gaccaataattagttattcaccttg

SEQ ID NO: 240 - DNA, 25 bp, homo sapiens
caacgagcaagaatttctttagcaa

SEQ ID NO: 241 - DNA, 25 bp, homo sapiens
gctagaccaataattagttattcac

SEQ ID NO: 242 - DNA, 25 bp, homo sapiens
acagagaatcctatgtacttgagat

SEQ ID NO: 243 - DNA, 25 bp, homo sapiens
gtgtgattgatagtaaccttactta

SEQ ID NO: 244 - DNA, 25 bp, homo sapiens
taatttaatttccattttcttttta

SEQ ID NO: 245 - DNA, 25 bp, homo sapiens
caaatcagcatctttgtatactgct

SEQ ID NO: 246 - DNA, 25 bp, homo sapiens
tttccattttctttttagagcagta

SEQ ID NO: 247 - DNA, 25 bp, homo sapiens
aataaatacaaatcagcatctttgt

SEQ ID NO: 248 - DNA, 25 bp, homo sapiens
agagcagtatacaaagatgctgatt

SEQ ID NO: 249 - DNA, 25 bp, homo sapiens
caaaaggagagtctaataaatacaa

SEQ ID NO: 250 - DNA, 25 bp, homo sapiens
caaagatgctgatttgtatttatta

SEQ ID NO: 251 - DNA, 25 bp, homo sapiens
acatctaggtatccaaaaggagagt

SEQ ID NO: 252 - DNA, 25 bp, homo sapiens
gctgatttgtatttattagactctc

SEQ ID NO: 253 - DNA, 25 bp, homo sapiens
tgttaaaacatctaggtatccaaaa

SEQ ID NO: 254 - DNA, 25 bp, homo sapiens
ccttttggatacctagatgttttaa

SEQ ID NO: 255 - DNA, 25 bp, homo sapiens
atacctttcaaatatttctttttct

SEQ ID NO: 256 - DNA, 33 bp, homo sapiens
ccttttggatacctagatgttttaacagaaaaa

SEQ ID NO: 257 - DNA, 35 bp, homo sapiens
gtaaggtattcaaagaacatacctttcaaatattt

SEQ ID NO: 258 - DNA, 36 bp, homo sapiens
cctagatgttttaacagaaaaagaaatatttgaaag

SEQ ID NO: 259 - DNA, 25 bp, homo sapiens
ataagtaaggtattcaaagaacata

SEQ ID NO: 260 - DNA, 25 bp, homo sapiens
agaaaaagaaatatttgaaaggtat

SEQ ID NO: 261 - DNA, 25 bp, homo sapiens
caatataagtaaggtattcaaagaa

SEQ ID NO: 262 - DNA, 25 bp, homo sapiens
tgtgtctgtaaactgatggctaaca

SEQ ID NO: 263 - DNA, 25 bp, homo sapiens
ccattttagaagtgaccaaaatcct

SEQ ID NO: 264 - DNA, 25 bp, homo sapiens
actaggattttggtcacttctaaaa

SEQ ID NO: 265 - DNA, 25 bp, homo sapiens
gagataaagtctggctgtagatttt

SEQ ID NO: 266 - DNA, 25 bp, homo sapiens
ttaaagaaagctgacaaaatattaa

SEQ ID NO: 267 - DNA, 25 bp, homo sapiens
aaaatagctgctaccttcatgcaaa

SEQ ID NO: 268 - DNA, 25 bp, homo sapiens
cattttcagaactccaaaatctaca

SEQ ID NO: 269 - DNA, 25 bp, homo sapiens
ccatgagttttgagctaaagtctgg

SEQ ID NO: 270 - DNA, 25 bp, homo sapiens
tccaaaatctacagccagactttag

SEQ ID NO: 271 - DNA, 25 bp, homo sapiens
ttggtcgaaagaatcacatcccatg

SEQ ID NO: 272 - DNA, 25 bp, homo sapiens
tttagctcaaaactcatgggatgtg

SEQ ID NO: 273 - DNA, 25 bp, homo sapiens
tactgcactaaattggtcgaaagaa

SEQ ID NO: 274 - DNA, 25 bp, homo sapiens
gaccaatttagtgcagaaagaagaa

SEQ ID NO: 275 - DNA, 25 bp, homo sapiens
gtaaggtctcagttaggattgaatt

SEQ ID NO: 276 - DNA, 25 bp, homo sapiens
ttcgaccaatttagtgcagaaagaa

SEQ ID NO: 277 - DNA, 25 bp, homo sapiens
tgagaaacggtgtaaggtctcagtt

SEQ ID NO: 278 - DNA, 25 bp, homo sapiens
actgagaccttacaccgtttctcat

SEQ ID NO: 279 - DNA, 25 bp, homo sapiens
caggagacaggagcatctccttcta

SEQ ID NO: 280 - DNA, 25 bp, homo sapiens
atgctcctgtctcctggacagaaac

SEQ ID NO: 281 - DNA, 32 bp, homo sapiens
ctctccagtctgtttaaaagattgtttttttg

SEQ ID NO: 282 - DNA, 25 bp, homo sapiens
tgtctcctggacagaaacaaaaaaa

SEQ ID NO: 283 - DNA, 31 bp, homo sapiens
ctctccagtctgtttaaaagattgttttttt

SEQ ID NO: 284 - DNA, 25 bp, homo sapiens
ctgtctcctggacagaaacaaaaaa

SEQ ID NO: 285 - DNA, 26 bp, homo sapiens
ccagtctgtttaaaagattgtttttt

SEQ ID NO: 286 - DNA, 25 bp, homo sapiens
gtctgtttaaaagattgtttttttg

SEQ ID NO: 287 - DNA, 25 bp, homo sapiens
agtctgtttaaaagattgttttttg

SEQ ID NO: 288 - DNA, 25 bp, homo sapiens
cctgtctcctggacagaaacaaaaa

SEQ ID NO: 289 - DNA, 25 bp, homo sapiens
ctctccagtctgtttaaaagattgt

SEQ ID NO: 290 - DNA, 28 bp, homo sapiens
gctcctgtctcctggacagaaacaaaaa

SEQ ID NO: 291 - DNA, 28 bp, homo sapiens
caaactctccagtctgtttaaaagattg

SEQ ID NO: 292 - DNA, 25 bp, homo sapiens
ttcaatcctaactgagaccttacac

SEQ ID NO: 293 - DNA, 25 bp, homo sapiens
ggagcatctccttctaatgagaaac

SEQ ID NO: 294 - DNA, 25 bp, homo sapiens
tgtctcctggacagaaacaaaaaaa

SEQ ID NO: 295 - DNA, 25 bp, homo sapiens
aactctccagtctgtttaaaagatt

SEQ ID NO: 296 - DNA, 25 bp, homo sapiens
tattctcaatccaatcaactctata

SEQ ID NO: 297 - DNA, 25 bp, homo sapiens
gtcttttgcacaatggaaaattttc

SEQ ID NO: 298 - DNA, 25 bp, homo sapiens
tctcaatccaatcaactctatacga

SEQ ID NO: 299 - DNA, 25 bp, homo sapiens
ggagtcttttgcacaatggaaaatt

SEQ ID NO: 300 - DNA, 25 bp, homo sapiens
tcccttacaaatgaatggcatcgaa

SEQ ID NO: 301 - DNA, 25 bp, homo sapiens
tctaaaggctcatcagaatcctctt

SEQ ID NO: 302 - DNA, 25 bp, homo sapiens
agaggcgatactgcctcgcatcagc

SEQ ID NO: 303 - DNA, 25 bp, homo sapiens
tgaagcgtggggccagtgctgatca

SEQ ID NO: 304 - DNA, 25 bp, homo sapiens
cagcactggccccacgcttcaggca

SEQ ID NO: 305 - DNA, 25 bp, homo sapiens
aggttcaggacagactgcctccttc

SEQ ID NO: 306 - DNA, 25 bp, homo sapiens
agagcataccagcagtgactacatg

SEQ ID NO: 307 - DNA, 30 bp, homo sapiens
gtggacagtaatatatcgaaggtatgtgtt

SEQ ID NO: 308 - DNA, 25 bp, homo sapiens
aatttttgtgctaatttggtgctta

SEQ ID NO: 309 - DNA, 25 bp, homo sapiens
attcttacctctgccagaaaaatta

SEQ ID NO: 310 - DNA, 25 bp, homo sapiens
cattccaggtggctgcttctttggt

SEQ ID NO: 311 - DNA, 25 bp, homo sapiens
gaatactcactttccaaggagccac

SEQ ID NO: 312 - DNA, 25 bp, homo sapiens
tgtgctgtggctccttggaaagtga

SEQ ID NO: 313 - DNA, 25 bp, homo sapiens
aatctacacaataggacatggaata

SEQ ID NO: 314 - DNA, 25 bp, homo sapiens
attaacgatttcctatttgctttac

SEQ ID NO: 315 - DNA, 25 bp, homo sapiens
ttccctttgtcttgaagaggagtgc

SEQ ID NO: 316 - DNA, 25 bp, homo sapiens
ctatttgctttacagcactcctctt

SEQ ID NO: 317 - DNA, 25 bp, homo sapiens
ctatgagtactattccctttgtctt

SEQ ID NO: 318 - DNA, 25 bp, homo sapiens
cgaaaattttacaccacaaaatgtt

SEQ ID NO: 319 - DNA, 25 bp, homo sapiens
aaagtacctgctttcgacgtgttga

SEQ ID NO: 320 - DNA, 25 bp, homo sapiens
gtgattatcaccagcaccagttcgt

SEQ ID NO: 321 - DNA, 25 bp, homo sapiens
cccacgtaaatgtaaaacacataat

SEQ ID NO: 322 - DNA, 25 bp, homo sapiens
ctggtgcatactctaatcacagtgt

SEQ ID NO: 323 - DNA, 25 bp, homo sapiens
taacattttgtggtgtaaaattttc

SEQ ID NO: 324 - DNA, 25 bp, homo sapiens
ctcttaccatatttgacttcatcca

SEQ ID NO: 325 - DNA, 25 bp, homo sapiens
cttaacggtacttatttttacatac

SEQ ID NO: 326 - DNA, 25 bp, homo sapiens
tcttaccatatttgacttcatccag

SEQ ID NO: 327 - DNA, 25 bp, homo sapiens
acttaacggtacttatttttacata

SEQ ID NO: 328 - DNA, 25 bp, homo sapiens
accaacatgttttctttgatcttac

SEQ ID NO: 329 - DNA, 25 bp, homo sapiens
agctccaatcacaattaataacaac

SEQ ID NO: 330 - DNA, 25 bp, homo sapiens
ttgatcttacagttgttattaattg

SEQ ID NO: 331 - DNA, 25 bp, homo sapiens
gcgacaactgctatggctccaatca

SEQ ID NO: 332 - DNA, 25 bp, homo sapiens
ttgttattaattgtgattggagcta

SEQ ID NO: 333 - DNA, 25 bp, homo sapiens
gggttctaaaactgcgacaactgct

SEQ ID NO: 334 - DNA, 25 bp, homo sapiens
tagcagttgtcgcagttttacaacc

SEQ ID NO: 335 - DNA, 25 bp, homo sapiens
ggcactgttgcaacaaagatgtagg

SEQ ID NO: 336 - DNA, 25 bp, homo sapiens
agcagtagtcgcagttttacaaccc

SEQ ID NO: 337 - DNA, 25 bp, homo sapiens
gcactgttgcaacaaagatgtaggg

SEQ ID NO: 338 - DNA, 25 bp, homo sapiens
catctttgttgcaacagtgccagtg

SEQ ID NO: 339 - DNA, 28 bp, homo sapiens
atatgcacacaacataataaaagccact

SEQ ID NO: 340 - DNA, 25 bp, homo sapiens
aacataataaaagccactatcactg

SEQ ID NO: 341 - DNA, 25 bp, homo sapiens
gctctcaacataataaaagccactg

SEQ ID NO: 342 - DNA, 25 bp, homo sapiens
gcaactcaaacaactggaatctgaa

SEQ ID NO: 343 - DNA, 25 bp, homo sapiens
tgagtatcgcacattcactgtcata

SEQ ID NO: 344 - DNA, 25 bp, homo sapiens
acattttgtgtttatgttatttgca

SEQ ID NO: 345 - DNA, 25 bp, homo sapiens
ctgtgaaatatttccatagaaaaca

SEQ ID NO: 346 - DNA, 25 bp, homo sapiens
ctgtcaacactgcgctggttccaaa

SEQ ID NO: 347 - DNA, 25 bp, homo sapiens
gatgacaaaaatcatttctattctc

SEQ ID NO: 348 - DNA, 25 bp, homo sapiens
cactcatcttgttacaagcttaaaa

SEQ ID NO: 349 - DNA, 25 bp, homo sapiens
ccgaaggcacgaagtgtccatagtc

SEQ ID NO: 350 - DNA, 25 bp, homo sapiens
aagcttaaaaggactatggacactt

SEQ ID NO: 351 - DNA, 25 bp, homo sapiens
aagtaaggctgccgtccgaaggcac

SEQ ID NO: 352 - DNA, 25 bp, homo sapiens
agcttaaaaggactatggacacttc

SEQ ID NO: 353 - DNA, 25 bp, homo sapiens
aaagtaaggctaccgtccgaaggca

SEQ ID NO: 354 - DNA, 25 bp, homo sapiens
aggactatggacacttcgtgccttc

SEQ ID NO: 355 - DNA, 25 bp, homo sapiens
agagtttcaaagtaaggctgccgtc

SEQ ID NO: 356 - DNA, 25 bp, homo sapiens
ctatggacacttcgtgccttcggac

SEQ ID NO: 357 - DNA, 25 bp, homo sapiens
gaacagagtttcaaagtaaggctgc

SEQ ID NO: 358 - DNA, 25 bp, homo sapiens
ggacggcagccttactttgaaactc

SEQ ID NO: 359 - DNA, 25 bp, homo sapiens
atgtaaattcagagctttgtggaac

SEQ ID NO: 360 - DNA, 25 bp, homo sapiens
gctctgaatttacatactgccaact

SEQ ID NO: 361 - DNA, 25 bp, homo sapiens
gcgcagtgttgacaggtacaagaac

SEQ ID NO: 362 - DNA, 25 bp, homo sapiens
tacatactgccaactggttcttgta

SEQ ID NO: 363 - DNA, 25 bp, homo sapiens
tttggaaccagcgcagtgttgacag

SEQ ID NO: 364 - DNA, 25 bp, homo sapiens
catactgccaactggttcttgtacc

SEQ ID NO: 365 - DNA, 25 bp, homo sapiens
catttggaaccagcgcagtgttgac

SEQ ID NO: 366 - DNA, 25 bp, homo sapiens
gttcttgtacctgtcaacactgcgc

SEQ ID NO: 367 - DNA, 25 bp, homo sapiens
atcatttctattctcatttggaacc

SEQ ID NO: 368 - DNA, 25 bp, homo sapiens
tacctgtcaacactgcgctggttcc

SEQ ID NO: 369 - DNA, 25 bp, homo sapiens
gacaaaaatcatttctattctcatt

SEQ ID NO: 370 - DNA, 25 bp, homo sapiens
tcatttacgtcttttgtgcatctat

SEQ ID NO: 371 - DNA, 25 bp, homo sapiens
taccaactcttccttctccttctcc

SEQ ID NO: 372 - DNA, 25 bp, homo sapiens
gcagtgggctgtaaactccagcata

SEQ ID NO: 373 - DNA, 25 bp, homo sapiens
ataagacttaccaagctatccacat

SEQ ID NO: 374 - DNA, 25 bp, homo sapiens
aatgttgttatttttatttcagatg

SEQ ID NO: 375 - DNA, 25 bp, homo sapiens
aacttaaagactcggctcacagatc

SEQ ID NO: 376 - DNA, 25 bp, homo sapiens
tttatttcagatgcgatctgtgagc

SEQ ID NO: 377 - DNA, 25 bp, homo sapiens
ggcatgtcaatgaacttaaagactc

SEQ ID NO: 378 - DNA, 25 bp, homo sapiens
acatgccaacagaaggtaaacctac

SEQ ID NO: 379 - DNA, 25 bp, homo sapiens
attcttgtatggtttggttgacttg

SEQ ID NO: 380 - DNA, 31 bp, homo sapiens
caactctcgaaagttatgattattgagaatt

SEQ ID NO: 381 - DNA, 25 bp, homo sapiens
ccagatgtcatctttcttcacgtgt

SEQ ID NO: 382 - DNA, 25 bp, homo sapiens
gattattgagaattcacacgtgaag

SEQ ID NO: 383 - DNA, 25 bp, homo sapiens
ccccctgagggccagatgtcatctt

SEQ ID NO: 384 - DNA, 25 bp, homo sapiens
ttattgagaattcacacgtgaagaa

SEQ ID NO: 385 - DNA, 25 bp, homo sapiens
ccctgagggccagatgtcatctttc

SEQ ID NO: 386 - DNA, 25 bp, homo sapiens
ttattgagaattcacacgtgaagga

SEQ ID NO: 387 - DNA, 25 bp, homo sapiens
ccccctgagggccagatgtcatctc

SEQ ID NO: 388 - DNA, 25 bp, homo sapiens
gtcaaagatctcacagcaaaataca

SEQ ID NO: 389 - DNA, 25 bp, homo sapiens
ctctaatatggcatttccaccttct

SEQ ID NO: 390 - DNA, 25 bp, homo sapiens
tggaaatgccatattagagaacatt

SEQ ID NO: 391 - DNA, 25 bp, homo sapiens
ctggccaggacttattgagaaggaa

SEQ ID NO: 392 - DNA, 25 bp, homo sapiens
ctcaataagtcctagccagagggtg

SEQ ID NO: 393 - DNA, 25 bp, homo sapiens
taacaaagcaagcagtgttcaaatc

SEQ ID NO: 394 - DNA, 25 bp, homo sapiens
tccatctgttgcagtattaaaatgg

SEQ ID NO: 395 - DNA, 25 bp, homo sapiens
catttcctttcagggtgtcttactc

SEQ ID NO: 396 - DNA, 25 bp, homo sapiens
gtgatcccatcacttttaccttata

SEQ ID NO: 397 - DNA, 25 bp, homo sapiens
atccagttcttcccaagaggcccac

SEQ ID NO: 398 - DNA, 25 bp, homo sapiens
tgggcctcttgggaagaactggatc

SEQ ID NO: 399 - DNA, 25 bp, homo sapiens
aagctgataacaaagtactcttccc

SEQ ID NO: 400 - DNA, 25 bp, homo sapiens
agagtactttgttatcagctttttt

SEQ ID NO: 401 - DNA, 25 bp, homo sapiens
ttcagtgttcagtagtctcaaaaaa

SEQ ID NO: 402 - DNA, 25 bp, homo sapiens
tagagaacatttccttctcaataag

SEQ ID NO: 403 - DNA, 25 bp, homo sapiens
gttcaaatctcaccctctggccagg

SEQ ID NO: 404 - DNA, 25 bp, homo sapiens
catttccttctcaataagtcctggc

SEQ ID NO: 405 - DNA, 25 bp, homo sapiens
aagcagtgttcaaatctcaccctct

SEQ ID NO: 406 - DNA, 25 bp, homo sapiens
tttaccttataggtgggcctcttgg

SEQ ID NO: 407 - DNA, 25 bp, homo sapiens
agtactcttccctgatccagttctt

SEQ ID NO: 408 - DNA, 25 bp, homo sapiens
ggcctcttgggaagaactggatcag

SEQ ID NO: 409 - DNA, 25 bp, homo sapiens
aaaagctgataacaaagtactcttc

SEQ ID NO: 410 - DNA, 25 bp, homo sapiens
ttgggaagaactggatcagggaaga

SEQ ID NO: 411 - DNA, 31 bp, homo sapiens
cagtagtctcaaaaaagctgataacaaagta

SEQ ID NO: 412 - DNA, 25 bp, homo sapiens
gggaagaactggaacagggaagagt

SEQ ID NO: 413 - DNA, 25 bp, homo sapiens
agtctcaaaaaagctgataacaaag

SEQ ID NO: 414 - DNA, 25 bp, homo sapiens
gaattcacacgtgaagaaagatgac

SEQ ID NO: 415 - DNA, 25 bp, homo sapiens
gtcatttggccccctgagggccaga

SEQ ID NO: 416 - DNA, 25 bp, homo sapiens
ggatcagggaagattactttgttat

SEQ ID NO: 417 - DNA, 25 bp, homo sapiens
agtgttcagtagtctcaaaaaagct

SEQ ID NO: 418 - DNA, 25 bp, homo sapiens
gaacactgaaggagaaatccagatc

SEQ ID NO: 419 - DNA, 25 bp, homo sapiens
gttattgaatcccaagacacaccat

SEQ ID NO: 420 - DNA, 25 bp, homo sapiens
ttgggattcaataactttgcaacag

SEQ ID NO: 421 - DNA, 25 bp, homo sapiens
ggtatcactccaaaggctttcctcc

SEQ ID NO: 422 - DNA, 25 bp, homo sapiens
gggattcaataactttgcaacagtg

SEQ ID NO: 423 - DNA, 25 bp, homo sapiens
gtggtatcactccaaaggctttcct

SEQ ID NO: 424 - DNA, 25 bp, homo sapiens
gattcaataactttgcaacagtgga

SEQ ID NO: 425 - DNA, 25 bp, homo sapiens
ctgtggtatcactccaaaggctttc

SEQ ID NO: 426 - DNA, 25 bp, homo sapiens
gaaagcctttggagtgataccacag

SEQ ID NO: 427 - DNA, 25 bp, homo sapiens
ttttctggctaagtccttttgctca

SEQ ID NO: 428 - DNA, 33 bp, homo sapiens
ttcttcttttcttttttgctatagaaagtattt

SEQ ID NO: 429 - DNA, 32 bp, homo sapiens
ccaagttttttctaaatgttccagaaaaaata

SEQ ID NO: 430 - DNA, 39 bp, homo sapiens
tcttcttttcttttttgctatagaaagtatttatttttt

SEQ ID NO: 431 - DNA, 30 bp, homo sapiens
ccaagttttttctaaatgttccagaaaaaa

SEQ ID NO: 432 - DNA, 36 bp, homo sapiens
gaaagtatttattttttctggaacatttagaaaaaa

SEQ ID NO: 433 - DNA, 25 bp, homo sapiens
cactccactgttcatagggatccaa

SEQ ID NO: 434 - DNA, 39 bp, homo sapiens
gttattcatactttcttcttcttttcttttttgctatag

SEQ ID NO: 435 - DNA, 25 bp, homo sapiens
atcatttcagttagcagccttacct

SEQ ID NO: 436 - DNA, 25 bp, homo sapiens
aacagccatttccataggtcataga

SEQ ID NO: 437 - DNA, 25 bp, homo sapiens
atcgtactgccgcactttgttctct

(19) United States
(12) Patent Application Publication
(10) Pub. No.: US 2005/0048544 A1
(43) Pub. Date: Mar. 3, 2005

(54) Methods and compositions for determining whether a subject carries a cystic fibrosis transmembrane conductance regulator (CFTR) gene mutation

(75) Inventors: Phyllis Gardner, Stanford, CA (US); Iris Schrijver, San Jose, CA (US)

Correspondence Address: BOZICEVIC, FIELD & FRANCIS LLP, 1900 UNIVERSITY AVE, SUITE 200, EAST PALO ALTO, CA, 94303, US

(73) Assignee: The Board of Trustees of the Leland Stanford Junior University

(21) Appl. No.: 10/888,435

(22) Filed: Jul. 9, 2004

Related U.S. Application Data
(60) Provisional application No. 60/486,763, filed on Jul. 10, 2003.

(51) Int. Cl. C12Q 1/68; C12P 19/34

(52) U.S. Cl. 435/6; 435/91.2

(57) ABSTRACT

Methods are provided for determining whether a subject carries a CFTR gene mutation. In practicing the subject methods, an array comprising a plurality of CFTR gene mutation probes is contacted with a nucleic acid sample from the subject, and the presence of any resultant surface bound target nucleic acids is detected to determine whether the subject carries a CFTR gene mutation. In addition, reagents and kits thereof that find use in practicing the subject methods are provided.

### CROSS-REFERENCE TO RELATED APPLICATIONS

This application claims priority (pursuant to 35 U.S.C. § 119 (e)) to the filing date of the U.S. Provisional Patent Application Ser. No. 60/486,763 filed Jul. 10, 2003; the disclosure of which is herein incorporated by reference.

### INTRODUCTION

1. Field of the Invention

The field of this invention is Cystic Fibrosis, and particularly the detection of Cystic Fibrosis Transmembrane Conductance Regulator (CFTR) gene mutations.

2. Background of the Invention

There has been considerable interest in developing genetic tests for genes responsible for disorders such as cystic fibrosis. Major pathologies associated with cystic fibrosis occur in the lungs, pancreas, sweat glands, digestive and reproductive organs. The gene associated with cystic fibrosis, CFTR, is a large gene with complex mutation and polymorphism patterns that pose a significant challenge to existing genotyping strategies. The CFTR gene has 27 exons, which span over 250 kb of DNA. Over 1200 mutations of various types (transitions, transversions, insertions, deletions and numerous polymorphisms) have been described.

Because the characterized CFTR mutations are widely distributed throughout the gene, existing genotyping assays focus only on the most common mutations. Some methods rely on using PCR to amplify regions surrounding mutations of interest and then characterizing the amplification products in a second analysis step, such as restriction fragment sizing, allele specific oligonucleotide hybridization, denaturing gradient gel electrophoresis, and single stranded conformational analysis. Alternatively, mutations have been analyzed using primers designed to amplify selectively mutant or wildtype sequences.

The American College of Medical Genetics (ACMG) and the American College of Obstetricians and Gynecologists (ACOG) have recently made a joint recommendation (Grody et al., (2001.) Genetics in Medicine 3: 149-154.) advising that CF carrier screening be made available to all ethnic and racial groups after appropriate education and with informed consent. It is anticipated that CF testing for at least 25 mutations (to detect only those mutations that have a frequency of at least 0.1% among CF patients in the US) will now be offered to all expecting couples and to those contemplating pregnancy.

While assays for CFTR mutations have been developed, there is continued interest in the identification of new assay formats. Of particular interest would be the development of a simple, cost effective and sensitive assay that can rapidly screen for the presence of a large number of different CFTR mutations. The present invention satisfies this need.

Relevant Literature

United States Patents of interest include: U.S. Pat. Nos. 6,027,880; 5,981,178; 6,001,588; 5,407,776; and 5,660,998. Also of interest are EP 0928832 and WO 94/08047.

### SUMMARY OF THE INVENTION

Methods are provided for determining whether a subject carries a CFTR gene mutation. In practicing the subject methods, an array comprising a plurality of CFTR gene mutation probes is contacted with a nucleic acid sample from the subject, and the presence of any resultant surface bound duplex nucleic acids is detected to determine whether the subject carries a CFTR gene mutation. In addition, reagents and kits thereof that find use in practicing the subject methods are provided.

### BRIEF DESCRIPTION OF THE FIGURES

FIG. 1: APEX analysis at mutation site 2183AA>G. Each numbered row represents the analysis of an individual patient sample. The row presents two sets of four-channel fluorescent images representing the bases adenine (A), thymine (T), guanosine (G), and cytosine (C) respectively for the sense strand (upper) and antisense strand (lower). The histograms to the right of the fluorescent images are of the fluorescent intensities of the four channels at the mutation analysis site. The letters to the right of the histogram represent the base(s) identified on each strand. Row 3 presents the results of heterozygous target DNA derived from a CF patient (WT/2183M>G). In this case, the sense strand is extended by both the wild type (WT) complementary target sequence base A and the base G complementary for the mutation, while the antisense strand is extended by the WT base T and the base C complementary for the mutation. Row 4 contains the results of normal DNA derived from a non-CF individual at the target sequence (WT/WT), with the expected WT base A in the sense channel and WT base T in the antisense channel. Row 5 contains the results of a homozygous target DNA derived from a CF patient (2183AA>G/2183AA>G), with the base G complementary for the mutation in the sense channel and the base C complementary for the mutation in the antisense channel.

FIG. 2: APEX analysis at mutation site ΔF508: Three patient samples, one each with ΔF508/DF508, WT/ΔF508, and WT/WT are presented. The results are presented as described in FIG. 1. Row 3 (upper) contains the results of homozygous target DNA derived from a CF patient (ΔF508/ΔF508). In this case, both the sense and the antisense strands are extended by the base T complementary in both strands for the mutation. Row 10 (center) contains the results of heterozygous target DNA from a CF patient (WT/ΔF508). In this case, the sense strand is extended by both the WT base C and base T complementary for the mutation, while the antisense strand is extended by the WT base A and the base T complementary for the mutation. Row 11 contains the results from a non-CF individual (WT/WT), in which the sense strand is extended by the WT base C, and the antisense strand is extended by the WT base A.

FIG. 3: APEX analysis at mutation sites G85E, 3849+10 kbC>T, 2789+5G>A. Three representative normal control versus heterozygous patient samples are shown for the mutation sites G85E (upper), 3849+10 kbC>T (middle), and 2789+5G>A (lower). These single nucleotide substitution mutations, the first encoding for amino acid change in exon 3 and second two encoding for sequence changes in introns 19 and 14 respectively, are prevalent in the Hispanic population. The results are presented as described in FIGS. 1. For G85E, Row 3 contains the results of a normal control DNA sample (WT/WT), with the sense strand extended by the WT base G and the antisense strand extended by the WT base C. Row 4 contains the results from a CF patient heterozygous at this site (WT/G85E). In this case the sense strand is extended both by the WT base G and the base A complementary for the mutation, while the antisense strand is extended by the WT sequence C and the base T complementary for the mutation. For 3849+10 kbC>T (middle), row 7 contains results from normal control DNA (WT/WT), with the sense strand extended by WT base C and the antisense strand extended by WT base G, while row 8 contains results from a CF patient heterozygous at this site (WT/3829+10 kbC>T). In this case, the sense strand is extended both by the WT base C and the base T complementary for the mutation, while the antisense strand is extended by WT base G and the base A complementary for the mutation. For 2789+5G>A (lower), row 19 contains results from normal control DNA (WT/WT), with the sense strand extended by the WT base G and the antisense strand extended by the WT sequence C, while row 20 contains the results from a CF patient heterozygous at this site (WT/2789+5G>A). In this case, the sense strand is extended both by the WT base G and the base A complementary for the mutation, and the antisense strand is extended by the WT base C and the base T complementary for the mutation.

FIG. 4: APEX analysis at mutation site IVS8-5T/7T/9T. Representative results for three patient samples are shown at mutation site IVS8-5T/7T/9T. This mutation site requires three pairs of allele specific primers for accurate identification. The first set of primers (A) consists of a sense strand that does not work reliably despite several iterations and thus should be discounted and an antisense strand predicted to give base C for 5T (−/C) and base A for either 7Tor 9T (−/A). The second set of primers (B) consists of a sense strand that elongates with a C only for 9T and an antisense strand that extends only with a C only for 5T. Thus the expected results for this set of primers is for 5T (−/C), for 7T (−/−) and for 9T (C/−). The third set of primers consists of a sense strand oligo that extends with A for 7T and T for 9T, while the antisense strand extends with C for 7T and A for 9T. Thus the expected set of results for the third set of primers is 5T (−/−), 7T (A/C), and 9T (T/A). Adding the three sets of results together, patient sample 30 can be identified as heterozygous 5T/7/T, patient sample 31 as heterozygous 7T/9T, and patient sample 32 as homozygous 9T/9T.

FIG. 5 provides a list of CFTR gene mutations of interest.

FIG. 6 provides a list of PCR primers employed to prepare a nucleic acid sample according to one embodiment of the subject invention.

FIG. 7 provides a list of CFTR probe sequences found on an array employed in one embodiment of the subject invention.

FIG. 8 provides a grid layout representation of an array employed in one embodiment of the subject invention.

### DESCRIPTION OF THE SPECIFIC EMBODIMENTS

Methods are provided for determining whether a subject carries a CFTR gene mutation. In practicing the subject methods, an array comprising a plurality of CFTR gene mutation probes is contacted with a nucleic acid sample from the subject, and the presence of any resultant surface bound target nucleic acids is detected to determine whether the subject carries a CFTR gene mutation. In addition, reagents and kits thereof that find use in practicing the subject methods are provided.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

As summarized above, the subject invention is directed to methods of determining whether a subject carries a CFTR gene mutation, as well as compositions of matter and kits thereof that find use in practicing the subject methods. In further describing the invention, the subject methods are described first in greater detail, followed by a review of representative applications in which the methods find use, as well as reagents and kits that find use in practicing the subject methods.

Methods of Determining Whether a Subject Carries a CFTR Gene Mutation

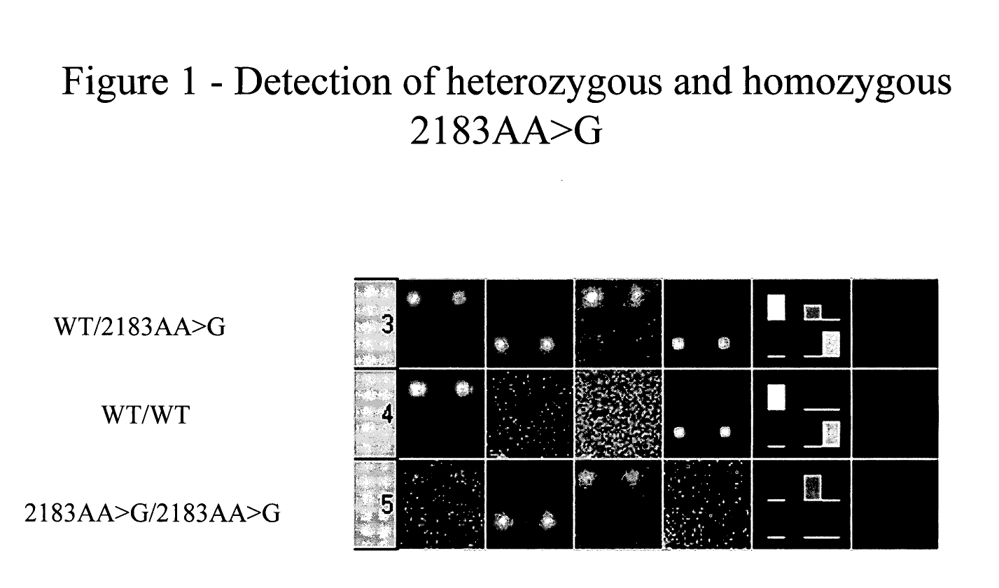

The subject invention provides methods of determining whether a patient or subject carries a CFTR gene mutation. By “carries” is meant whether a subject has a CFTR gene mutation, where the subject may be heterozygous or homozygous for the particular mutation and be considered to carry the mutation. By CFTR gene mutation is meant a mutation in the CFTR gene, i.e., the more than 250 kb of DNA including 27 exons that encode the CFTR gene product. The CFTR gene mutations that may be detected according to the subject invention may be deletion mutations, insertion mutations or point mutations, including substitution mutations. Of particular interest are CFTR gene mutations that result in an at least partially defective CFTR gene product, where the defective product may be manifested as the disease condition known as cystic fibrosis, particularly if the host or subject is homozygous for the particular CFTR gene mutation or heterozygous for two disease-causing mutations. Representative specific CFTR gene mutations of interest include, but are not limited to, those mutations listed in Table 1 appearing in FIG. 1.

In practicing methods of the subject invention, a host or subject is simultaneously screened for the presence of a plurality of different CFTR gene mutations. In many embodiments, the host or subject is simultaneously screened for the presence of at least 25 different mutations, usually at least about 40 different gene mutations and often at least about 50 different gene mutations, where in many embodiments the number of different gene mutations that are simultaneously screened is at least about 75, at least about 100, at least about 150, at least about 175, at least about 200 or more. In many embodiments, the collection of mutations for which a host or subject is simultaneously screened includes at least one mutation that appears in non-Caucasian individuals, where the number of such mutations may be at least about 5, at least about 10, at least about 20, at least about 25 or more. Representative non-Caucasian populations of interest include, but are not limited to: Hispanic, African, Asian, etc. In certain embodiments, the CFTR gene mutations that are screened or assayed in a given test include at least about 25 of the mutations listed in Table 1, such as at least about 50 of the mutations listed in Table 1, including at least about 75, at least about 100, at least about 125, at least about 150, at least about 175, at least about 200 or more, including of all of, the mutations listed in Table 1.

In one embodiment of the present invention, the host may be simultaneously screened for the presence of a plurality of CFTR gene mutations using any convenient protocol, so long as at least about 30, and typically at least about 50, of the mutations appearing in Table 1 are assayed. In such embodiments, representative protocols for screening a host for the presence of the CFTR gene mutations include, but are not limited to, array-based protocols, including those described in U.S. Pat. Nos. 6,027,880 and 5,981,178, the disclosures of which are herein incorporated by reference.

In certain embodiments of interest, an arrayed primer extension assay protocol (e.g., as described in Kurg et al., Genet. Test (2000) 4:1-7 and Tonisson et al., Microarray Biochip Technology (ed. Schena, Eaton Publishing, Natick Mass.) (2000) pp. 247-263) is employed to screen a subject for the presence of a plurality of different CFTR gene mutations. In such embodiments, an array of a plurality of distinct CFTR gene mutation specific probes is first contacted with a nucleic acid sample from the host or subject. The resultant sample contacted array is then subjected to primer extension reaction conditions in the presence of two or more, including four, distinguishably labeled dideoxynucleotides. The resultant surface bound labeled extended primers are then detected to determine the presence of a CFTR gene mutation in the host or subject from which the sample was obtained. Each of these steps is now described in greater detail below.

As summarized above, the first step of the protocol employed in these embodiments is to contact an array of a plurality of CFTR gene mutation probes with a nucleic acid sample from the host or subject being screened. The array employed in these embodiments includes a plurality of CFTR gene mutation probes immobilized on a surface of a solid substrate, where each given probe of the plurality is immobilized on the substrate surface at a known location, such that the location of a given probe can be used to identify the sequence or identity of that probe. Each given probe of the plurality is typically a single stranded nucleic acid, having a length of from about 10 to about 100 nt, including from about 15 to about 50 nt, e.g., from about 20 to about 30 nt, such as 25 nt. The arrays employed in the subject methods may vary with respect to configuration, e.g.,-shape of the substrate, composition of the substrate, arrangement of probes across the surface of the substrate, etc., as is known in the art. Numerous array configurations are known to those of skill in the art, and may be employed in the subject invention. Representative array configurations of interest include, but are not limited to, those described in U.S. Pat. Nos.: 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

As mentioned above, a feature of the arrays employed in this embodiment of the invention is that they include a plurality of different CFTR gene mutation probes. The total number of CFTR gene mutation probes that may be present on the surface of the array, i.e., the total number of CFTR gene mutations that may be represented on the array, may vary, but is in many embodiments at least about 25 or more, usually at least about 40 or more and often at least about 50 or more different gene mutations, where in many embodiments the number of different gene mutations that are represented on the array is at least about 75, at least about 100, at least about 150, at least about 175, at least about 200 or more. In certain embodiments, the CFTR gene mutations that are represented on the array in the form of probes include at least about 25 of the mutations listed in Table 1, such as at least about 50 of the mutations listed in Table 1, including at least about 75, at least about 100, at least about 125, at least about 150, at least about 175, at least about 200 or more, including of all of, the mutations listed in Table 1. In certain embodiments, the arrays employed in the subject methods include a pair of different probes for each given CFTR gene mutation represented on the array. Typically, the pair of probes corresponds to the sense and antisense strand of the CFTR gene region that includes the mutation of interest. Such a configuration is known in the art and described in Kurg et al., Genet. Test (2000) 4:1-7.

In certain embodiments, at least about 25 of the specific probes listed in Table 3, such as at least about 50 of the probes listed in Table 3, including at least about 75, at least about 100, at least about 125, at least about 150, at least about 175, at least about 200 or more, including of all of, the probes listed in Table 3 are present on the array that is employed to screen the nucleic acid sample.

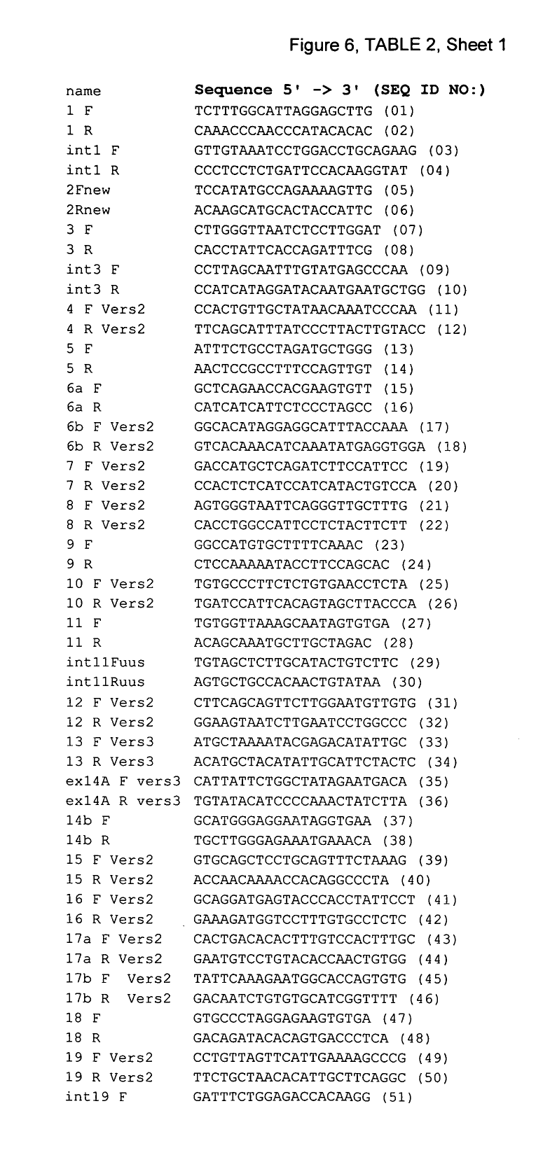

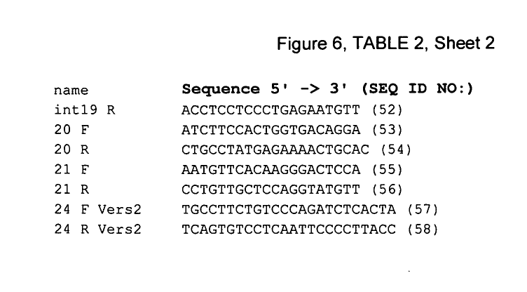

As summarized above, the first step in the subject methods is to contact a nucleic acid sample obtained from the host or subject being screened with the array to produce a sample contacted array. The nucleic acid sample is, in many embodiments, one that contains an amplified amount of fragmented CFTR gene nucleic acids, e.g., DNA or RNA, where in many embodiments the nucleic acid sample is a DNA sample. The nucleic acid sample is typically prepared from one or more cells or tissue harvested from a subject to be screened using standard protocols. Following harvesting of the initial nucleic acid sample, the sample is subjected to conditions that produce amplified amounts of the CFTR gene present in the sample. While any convenient protocol may be employed, in many embodiments the sample is contacted with a pair of primers that flank each region of interest of the CFTR gene, i.e., a pair of primers for each regions of interest of the CFTR gene, and then subjected to PCR conditions. This step results in the production of an amplified amount of nucleic acid for each particular region of location of the CFTR gene of interest. In certain embodiments, the primer pairs employed in this step include at least 1 primer pair appearing in Table 2 of FIG. 6, where in certain embodiments a plurality of primer pairs from Table 2 are employed, such as at least about 2 or more, including at least about 5 or more, 10 or more, 25 or more, including all of, the primer pairs appearing in Table 2. Amplification protocols that find use in such methods are well known to those of skill in the art.

The resultant nucleic acid composition that includes an amplified amount of the CFTR sequence is then fragmented to produce a fragmented CFTR gene sample. Fragmentation may be accomplished using any convenient protocol, where representative protocols of interest include both physical (e.g., shearing) and enzymatic protocols. In many embodiments, an enzymatic fragmentation protocol is employed, where the nucleic acid sample is contacted with one or more restriction endonucleases that cleave the CFTR gene nucleic acids into two or more fragments.

The resultant amplified fragmented CFTR gene nucleic acid sample is then contacted with the array under conditions sufficient to produce surface immobilized duplex nucleic acids between host or subject derived nucleic acids and any complementary probes present on the surface of the array. Typically, the sample is contacted with the array under stringent hybridization conditions. The term “stringent conditions” refers to conditions under which a probe will hybridize preferentially to its target subsequence, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. “Stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions set forth the conditions that determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions. Washing of the array following sample contact results in removal of any unbound nucleic acids from the surface of the array.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

Sample contact and washing of the array as described above results in the production of a sample contacted array, where the sample contacted array is characterized by the presence of surface bound duplex nucleic acids, generally at each position of the array where probe nucleic acids and target nucleic acids in the sample have sufficiently complementary sequences to hybridize with each other into duplex nucleic acids under the conditions of contact, e.g., stringent hybridization conditions.

Following production of the sample contacted array, as described above, the presence of any CFTR gene mutations in the assayed nucleic acid sample, and therefore the host genome from which the sample was prepared, is detected. Depending on the nature of the array employed and the detection protocol used, a number of different protocols may be employed for determining the presence of one or more CFTR gene mutations in the assayed nucleic acid sample. For example, in those embodiments where the array includes immobilized probes that specifically bind only to target nucleic acids generated from mutated genomic sequences, detection of surface bound duplex nucleic acids can be used directly to determine the presence of a CFTR gene mutation in the sample.

In many embodiments, the presence of any CFTR gene mutations is detected using a primer extension protocol, in which the surface bound probe component of the duplex nucleic acid acts as a primer which is extended in a template dependent primer extension reaction using the hybridized complement of the probe which is obtained from the patient derived nucleic acid sample as a template. In these embodiments, the sample-contacted array is contacted with primer extension reagents and maintained under primer extension conditions.

Primer extension reactions are well known to those of skill in the art. In this step of the subject methods, the sample-contacted array is contacted with a DNA polymerase under primer extension conditions sufficient to produce the desired primer extension molecules. DNA polymerases of interest include, but are not limited to, polymerases derived from E. coli, thermophilic bacteria, archaebacteria, phage, yeasts, Neurosporas, Drosophilas, primates and rodents. The DNA polymerase extends the probe “primer” according to the template to which it is hybridized in the presence of additional reagents which may include, but are not limited to: dNTPs; monovalent and divalent cations, e.g. KCl, MgCl₂; sulfhydryl reagents, e.g. dithiothreitol; and buffering agents, e.g. Tris-Cl.

In a particular embodiment of interest, the primer extension reaction of this step of the subject methods is carried out in the presence of at least two distinguishably labeled dideoxynucleotides or ddNTPs, and in many embodiments at least four distinguishably labeled dideoxynucleotide triphosphates (ddNTPs), e.g., ddATP, ddCTP, ddGTP and ddTTP, and in the absence of deoxynucleotide triphosphates (dNTPs).

Extension products that are produced as described above are typically labeled in the present methods. As such, the reagents employed in the subject primer extension reactions typically include a labeling reagent, where the labeling reagent is typically a labeled nucleotide, which may be labeled with a directly or indirectly detectable label. A directly detectable label is one that can be directly detected without the use of additional reagents, while an indirectly detectable label is one that is detectable by employing one or more additional reagents, e.g., where the label is a member of a signal producing system made up of two or more components. In many embodiments, the label is a directly detectable label, such as a fluorescent label, where the labeling reagent employed in such embodiments is a fluorescently tagged nucleotide(s), e.g., ddCTP. Fluorescent moieties which may be used to tag nucleotides for producing labeled probe nucleic acids include, but are not limited to: fluorescein, the cyanine dyes, such as Cy3, Cy5, Alexa 555, Bodipy 630/650, and the like. Other labels may also be employed as are known in the art.

In the primer extension reactions employed in the subject methods of these embodiments, the surface of the sample contacted array is maintained in a reaction mixture that includes the above-discussed reagents at a. sufficient temperature and for a sufficient period of time to produce the desired labeled probe “primer” extension products. Typically, this incubation temperature ranges from about 20° C. to about 75° C., usually from about 37° C. to about 65° C. The incubation time typically ranges from about 5 min to about 18 hr, usually from about 1 hr to about 12 hr.

Primer extension of any duplexes on the surface of the array substrate as described above results, in many embodiments, in the production of labeled primer extension products. In those embodiments where primer extension is carried out solely in the presence of distinguishably labeled ddNTPs, as described above, the primer extension reaction results in extension of the probe “templates” by one labeled nucleotide only.

Following production of labeled primer extension products, as described above, the presence of any labeled products is then detected, either qualitatively or quantitatively. Any convenient detection protocol may be employed, where the particular protocol that is used will necessarily depend on the particular array assay, e.g., the nature of the label employed. Representative detection protocols of interest include, but are not limited to, those described in U.S. Pat. Nos.: 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

Where the primer extension products are fluorescently labeled primer extension products, any convenient fluorescently labeled primer extension protocol may be employed. In many embodiments, a “scanner” is employed that is capable of scanning a surface of an array to detect the presence of labeled nucleic acids thereon. Representative scanner devices include, but are not limited to, those described in U.S. Pat. Nos. 5,585,639; 5,760,951; 5,763,870; 6,084,991; 6,222,664; 6,284,465; 6,329,196; 6,371,370 and 6,406,849. In certain embodiments of particular interest, the scanner employed is one that is capable of scanning an array for the presence of four different fluorescent labels, e.g., a four-channel scanner, such as the one disclosed in published U.S. Patent Application Ser. No. 20010003043; the disclosure of which is herein incorporated by reference.

The final step in these embodiments of the subject methods is to determine the presence of any CFTR gene mutations in the assayed sample, and therefore the host from which the sample was obtained, based on the results of the above surface immobilized duplex nucleic acid detection step. In this step of the subject methods, any detected labeled duplex nucleic acids, and specifically labeled extended primers, are employed to determine the presence of one or more CFTR gene mutations in the host from which the screened sample was obtained. This step is practiced by simply identifying the location on the array of the labeled duplex, and then identifying the probe (and typically sequence thereof) of the probe “primer” at that location which was extended and labeled. Identification of the probe provides the specific CFTR gene mutation that is present in the host from which the sample was obtained.

Using the above described protocols, the presence of one or more CFTR gene mutations in the genome of a given subject or host may be determined. In other words, whether or not a host carries one or more CFTR gene mutations may be determined using the subject methods. The subject methods may be employed to determine whether a host is homozygous or heterozygous for one or more CFTR gene mutations. A feature of the subject methods is that they provide for a highly sensitive assay for the presence of CFTR gene mutations across a broad population. For example, they provide for a sensitivity of at least about 60%, including at least about 65%, 70%, 75% or higher, e.g., 80%, 85%, 90% or higher, e.g., 95%, 97%, 99% or higher, in a plurality of different racial backgrounds, including Caucasian, Asian, Hispanic and African racial backgrounds.

Utility

The subject methods find use in a variety of different applications. In many embodiments, the above-obtained information is employed to diagnose a host, subject or patient with respect to whether or not they carry a particular CFTR gene mutation.

In yet other embodiments, the subject methods are employed to screen potential parents to determine whether they risk producing offspring that are homozygous for one or more CFTR mutations. In other words, the subject methods find using in genetic counseling applications, where prospective parents can be screened to determine there potential risk in producing a child that is homozygous for a CFTR gene mutation (or heterozygous for two disease causing mutations) and will suffer from a disease associated therewith, e.g., cystic fibrosis.

In yet other embodiments, the subject methods and compositions are employed to screen populations of individuals, e.g., to determine frequency of various mutations. For example, a select population of individuals, e.g., grouped together based on race, geographic region, etc., may be screened according to the subject invention to identify those mutations that appear in members of the population and/or determine the frequency at which such identified mutations appear in the population.

Reagents and Kits

Also provided are reagents and kits thereof for practicing one or more of the above-described methods. The subject reagents and kits thereof may vary greatly depending on the particular embodiment of the invention to be practiced. Reagents of interest include, but are not limited to: nucleic acid arrays (as described above); CFTR primers, e.g., for using in nucleic acid sample preparation, as described above, one or more uniquely labeled ddNTPs, DNA polymerases, various buffer mediums, e.g. hybridization and washing buffers, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

### I. Materials and Methods

A. Mutation Selection:

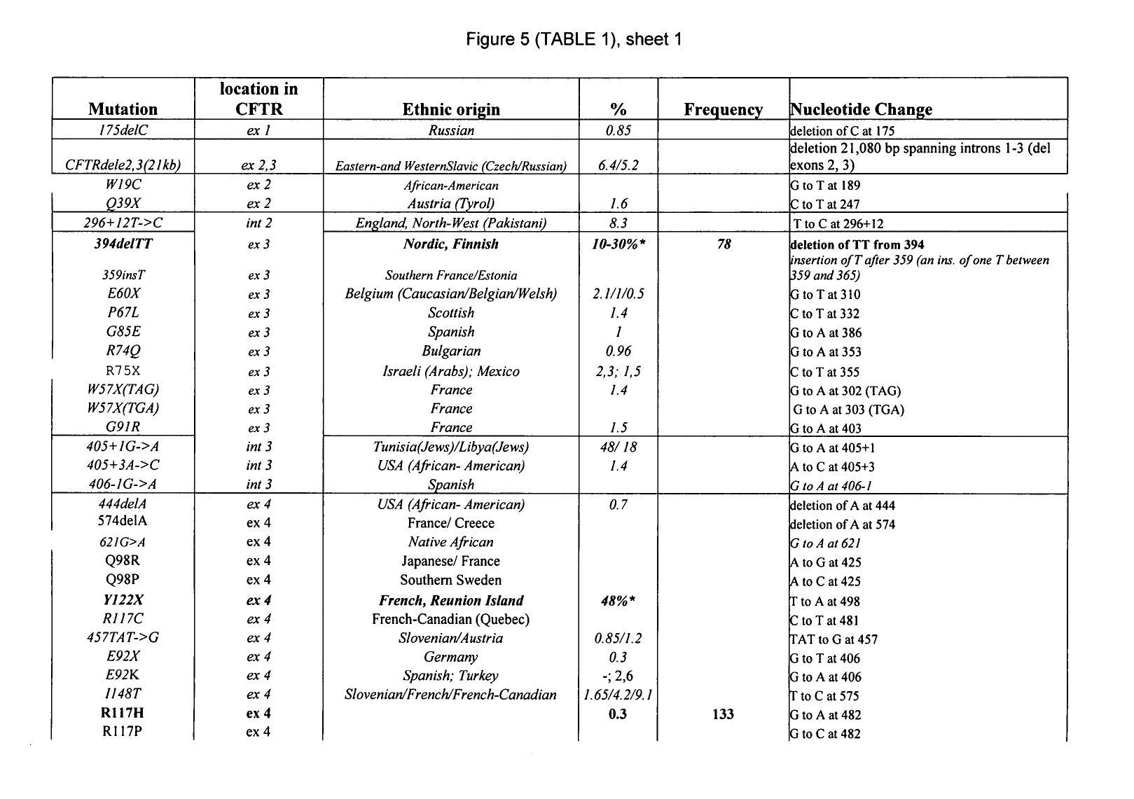

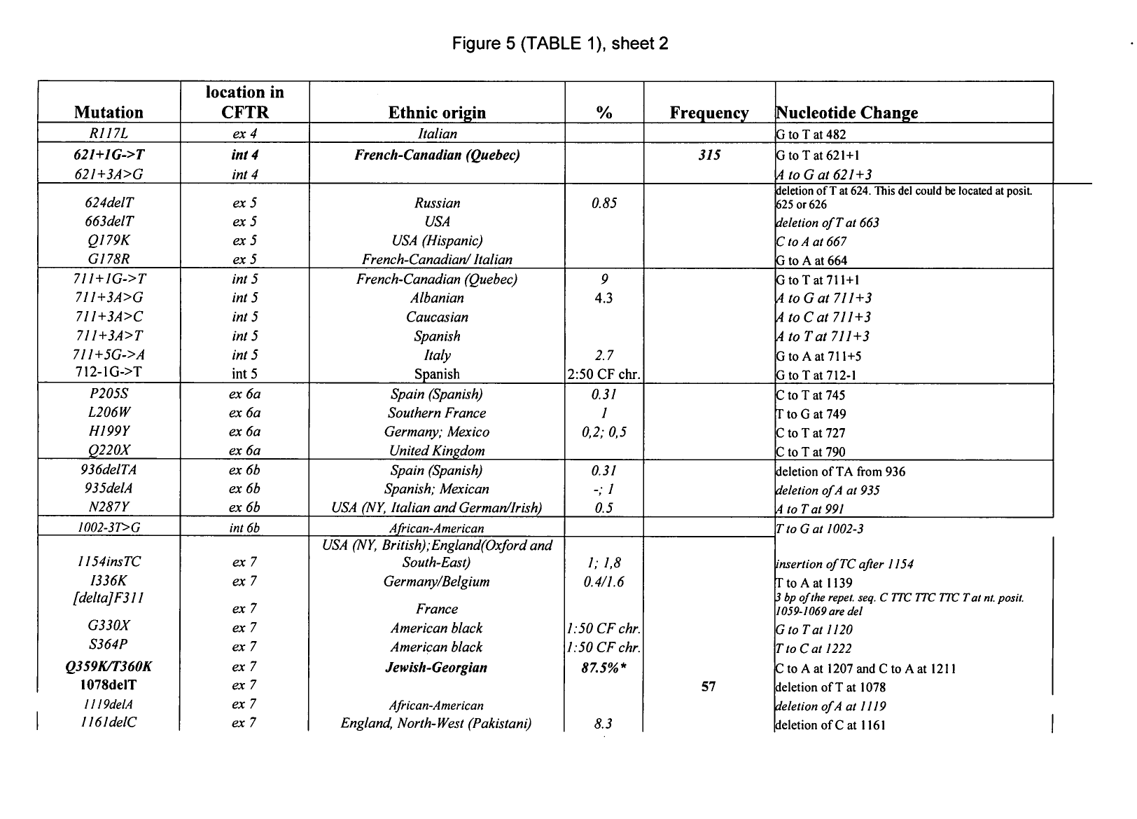

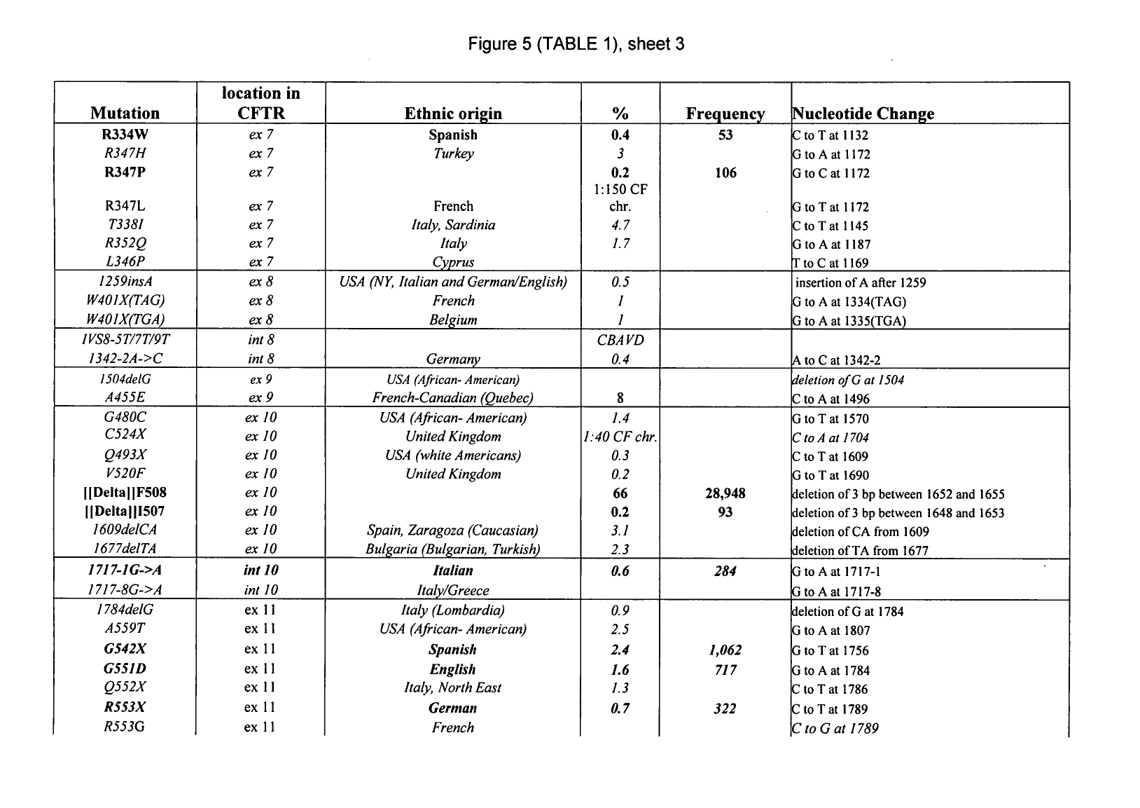

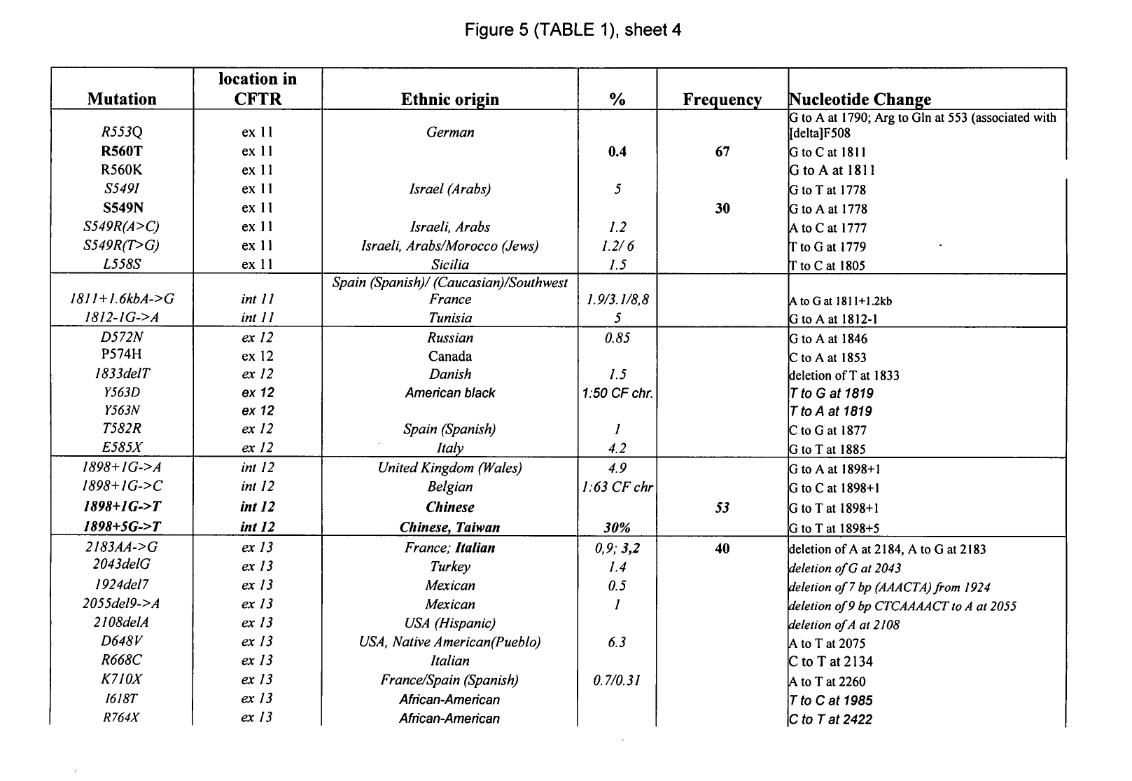

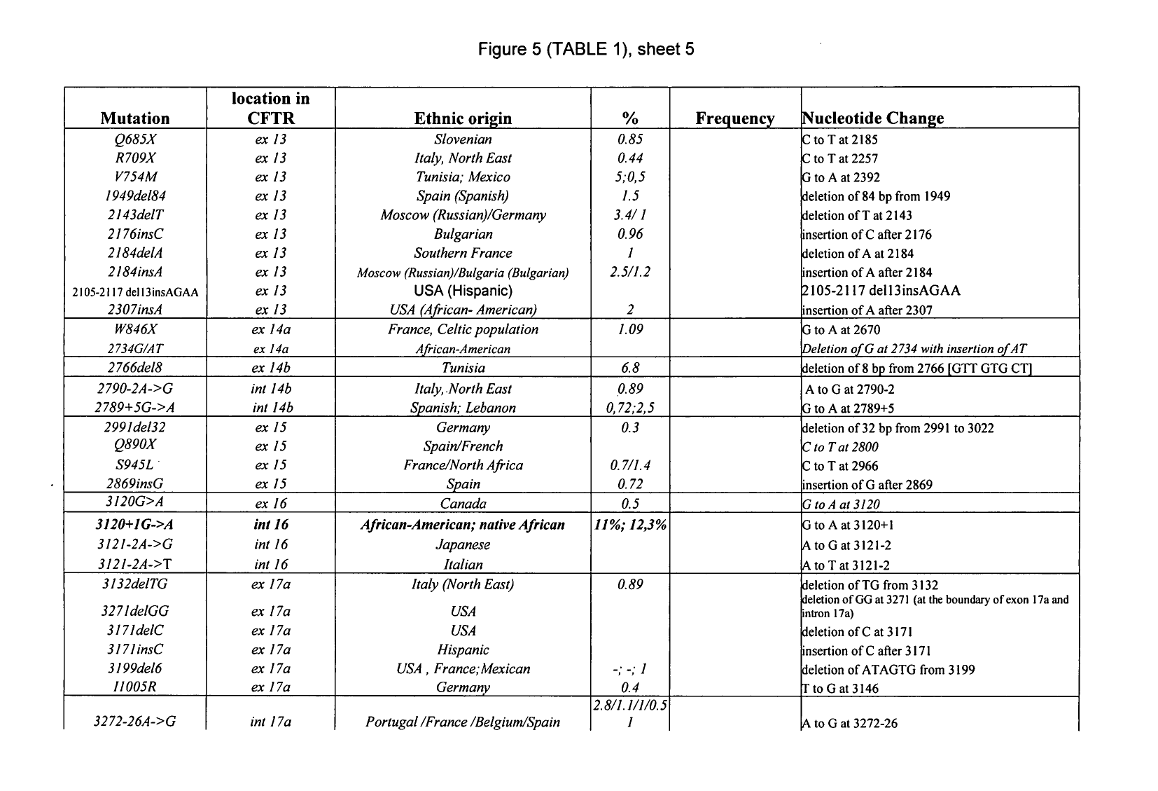

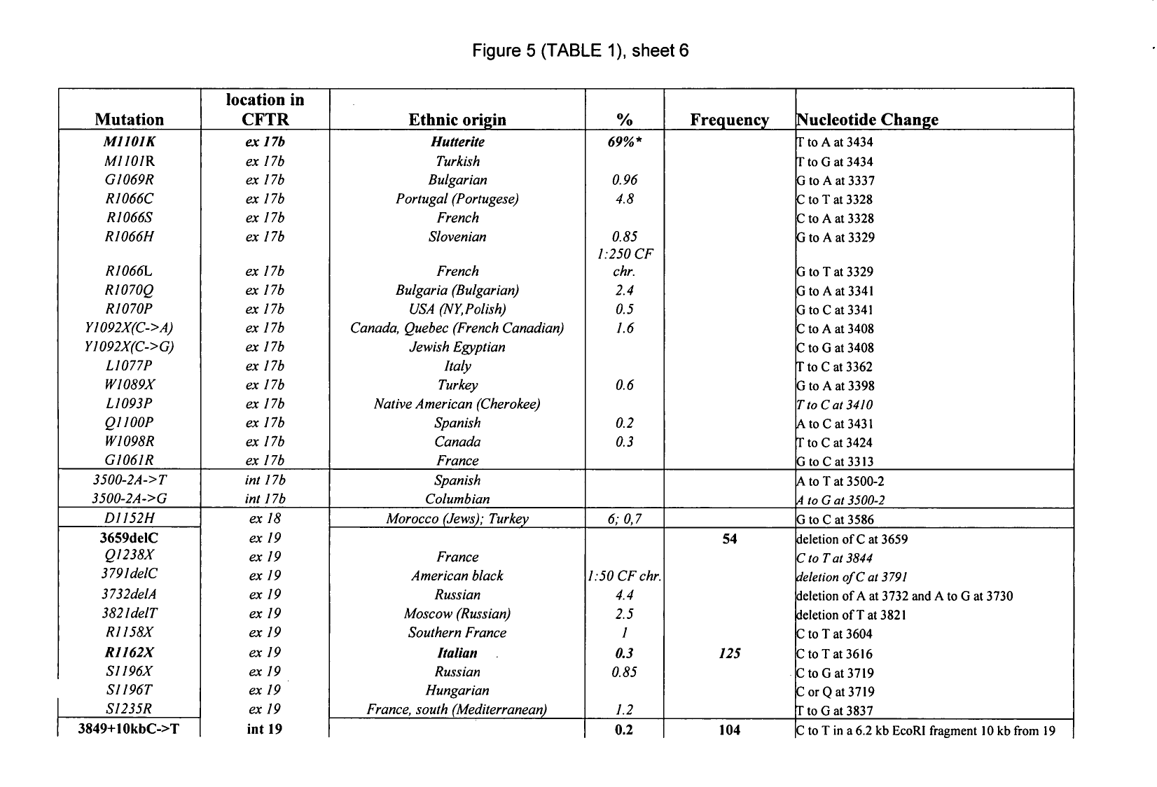

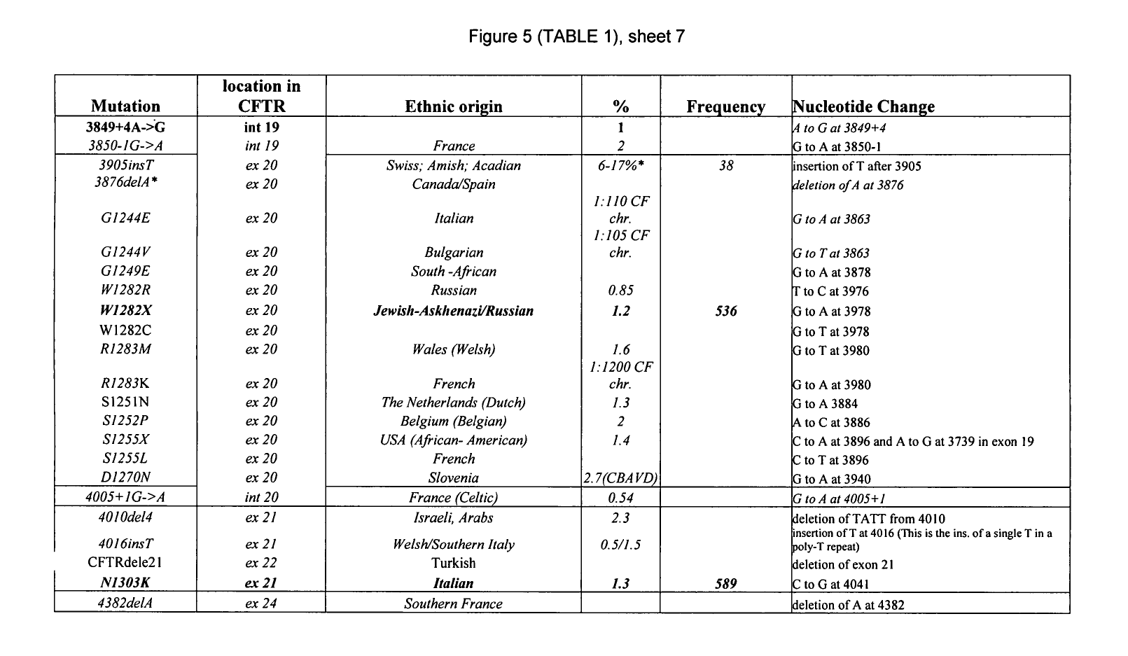

The mutations on the APEX microarray were selected from the information provided at the websites thate are prepared by placing “http//www.” before the following partial urls:
CF Genetic Analysis Consortium (1994) (genet.sickkids.on.ca/cftr/Table1.html), representing the most frequently screened mutations in Caucasians and those identified as recurring in specific Caucasian and non-Caucasian populations;
(genet.sickkids.on.a/cftr/rptTable3.html; and
(genet.sickkids.on.ca/cftr-cgi-bin/FullTable).
   The full set of mutations is listed in Table 1 in FIG. 5.
   B. Oligonucleotide Microchips:

Oligonucleotide primers were designed according to the wild-type CFTR gene sequence for both the sense and antisense directions. The 25 bp oligonucleotides with 6-carbon amino linkers at their 5′ end were obtained from MWG (Munich, Germany). Most scanning oligonucleotides were designed to scan 1 bp in the wild-type sequence, except in the case of deletions and insertions that have the same nucleotide in the 1 bp direction. In this case, we designed the oligo to extend further into the deletion or insertion to enable discrimination of the nucleotide change. For example

DeltaF508 sV1

| | |
|---|---|
| 5′ AGCCTGGCACCATTAAAGAAAATATCAT 3′ | (SEQ ID NOS:438-440) |
| 5′ TTTCCTGGATTATGCCTGGCACCATTAAAGAAAATATCATCTTTGGTGTTTCCTATGATGAATATAGATACAGA 3′ | |
| DeltaF508 as 3′ AACCACAAAGGATACTACTTATATC 5′ | |

in which the bold A represents a deliberate mismatch to avoid strong secondary structure and the italic CTT represents a deletion of three nucleotides. In case of the normal allele we will expect signals for the sense oligo in the cytosine (C) channel and for the antisense oligo in the adenine (A) channel (C/A). In case of the mutant allele we will detect signals for the sense oligo in the thymine (T) channel. The signal corresponding to the antisense oligo in will also appear in the T channel (T/T).

The microarray slides used for spotting the oligonucleotides have a dimension of 24×60 mm and are coated with 3-Aminopropyl-trimethoxysilane plus 1,4-Phenylenediisothiocyanate (Asper Biotech, Ltd., Tartu, Estonia). Primers were diluted to 50 μM in 100 mM carbonate buffer, pH 9.0, and spotted onto the activated surface with BioRad VersArray (BioRad Laboratories, Hercules, Calif.). The slides were blocked with 1% ammonia solution and stored at 4° C. until needed. Washing steps with 95° C. Milliq and 100 mM NaOH were performed prior to APEX reactions to reduce the background fluorescence and to avoid rehybridization of unbound oligonucleotides to the APEX slide.

C. Genomic and Synthetic Template Samples:

Where possible, native genomic DNA was collected from patients (50) with mutations represented on the chip. The samples were collected from Lucille Packard Children's Hospital (Stanford, Calif.), through a protocol approved by the Institute Review Board of Stanford University and with informed consent of all participants, or from the Molecular Diagnostics Centre of United Laboratories, Tartu University Clinics. Some DNA samples were a generous gift from Dr. Milan Macek Jr., Charles University, Institute of Biology & Med. Genetics, Department of Molecular Genetics, CF Center; Prague, Czech Republic. When it was not readily possible to obtain native genomic DNA samples with the screened mutations, synthetic 50 bp templates were designed according to the mutated CFTR sequence for both the sense and antisense directions (MWG, Germany). In this case, polyT tracts were designed at the 5′ end in order to minimize the possibility of the self-extensions and/or self-annealing of the synthetic templates.

D. Template Preparation:

The CFTR gene was amplified from genomic DNA in 29 amplicons with the primers listed in Table 2 in FIG. 6. The PCR reaction mixture (50 μL) was optimized with the following: 10× Taq DNA polymerase buffer; 2.5 mM MgCl₂ (Naxo, Estonia); 0.25 mM dNTP (MBI Fermentas, Vilnius, Lithuania) (20% fraction of dTTP was substituted with dUTP), 10 pmol primer stock, DNA (approximately 80 ng), SMART-Taq Hot DNA polymerase (3U) (Naxo, Estonia), and sterile deionized water. After amplification (MJ Research DNA Thermal Cycler; MJ Research, Inc., Waltham, Mass.), the amplification products were concentrated and purified using Jetquick spin columns (Genomed GmbH, Lohne, Germany). In a one-step reaction the functional inactivation of the traces of unincorporated dNTPs was achieved by addition of shrimp Alkaline Phosphatase (Amersham Pharmacia Biotech, Inc., Milwaukee, Wis.) and fragmentation of the PCR product was achieved by addition of thermolabile Uracil N-Glycosylase (Epicenter Technologies, Madison, Wis.) followed by heat treatment (Kurg et al., 2000).

E. Arrayed Primer Extension (APEX) Reactions:

The APEX mixture consisted of 32 μL fragmented product, 5U of Thermo Sequenase DNA polymerase (Amersham Pharmacia Biotech, Inc., Milwaukee, Wis.), 4 μL Thermo Sequenase reaction buffer (260 mM Tris-HCl, pH 9.5, 65 mM MgCl₂) (Amersham Pharmacia Biotech, Inc., Milwaukee, Wis.) and 1 μM final concentration of each fluorescently-labeled ddNTP-s: Cy5-ddUTP, Cy3-ddCTP, Texas Red-ddATP, Fluorescein-ddGTP, (PerkinElmer Life Sciences, Wellesley, Mass.). The DNA was first denatured at 95° C. for ten minutes. The enzyme and the dyes were immediately added to the DNA mixture, and the whole mixture was applied to prewarmed slides. The reaction was allowed to proceed for 10 minutes at 58° C., followed by washing once with 0.3% Alconox (Alconox, Inc.) and twice for 90 sec at 95° C. with MilliQ water. A droplet of antibleaching reagent (AntiFade SlowFade, Molecular Probes Europe BV, Leiden, The Netherlands) was applied to the slides before imaging.

F. Analysis:

The array images were captured by means of detector Genorama™ Quattrolmager 003 (Asper Biotech Ltd, Tartu, Estonia) at 20 μm resolution. The device combines a total internal reflection fluorescence (TIRF) based excitation mechanism with a charge coupled device (CCD) camera (Kurg et al., 2000). Sequence variants were identified using Genorama 3.0 genotyping software.

### II. Results

Known mutations of the CFTR gene were selected for a comprehensive diagnostic panel enabling CF carrier and disease detection across all racial and ethnic groups (Table 1). The mutations were selected from both the CF Genetic Analysis Consortium (1994) compiled from the screening of 43,849 chromosomes, as well as mutations studied in relatively small-size samples or isolated to specific ethnic populations. The frequencies of these mutations in the population vary considerably according to ethnicity and size of sample screened, but they represent the most common reported mutations across population groups to date. Mutations include several prominent in non-Caucasian races, including G542X, N1303K, 3849+10 kb, 2789+5G>A, 3876 delA, each prevalent in the Hispanic population, 3120+1G>A prevalent in the African American population, and 1898+5G>T prevalent in the Chinese population. These mutations come from 23 exons (exons 1-22 and exon 24) and from 13 introns (3,4,5,6,8, 10, 11, 12, 14, 16, 17, 19, 20). They include single nucleotide substitutions, technically the most easy to detect with the APEX reaction, as well as insertions, deletions, including the large deletion CFTRdele2,3(21 kb), and repeats, including the 5T/7T/9T repeats important in the disease congenital bilateral absence of the vas deferens (CBAVD). Sample DNA is amplified with 29 pairs of PCR primers (Table 2) encompassing the mutations, with PCR mixtures that include 20% substitution of dUTPs for dTTPs allowing for later fragmentation with uracil N-glycosylase (UNG) as described in Kurg et al., Genet. Test (2000) 4:1-7.

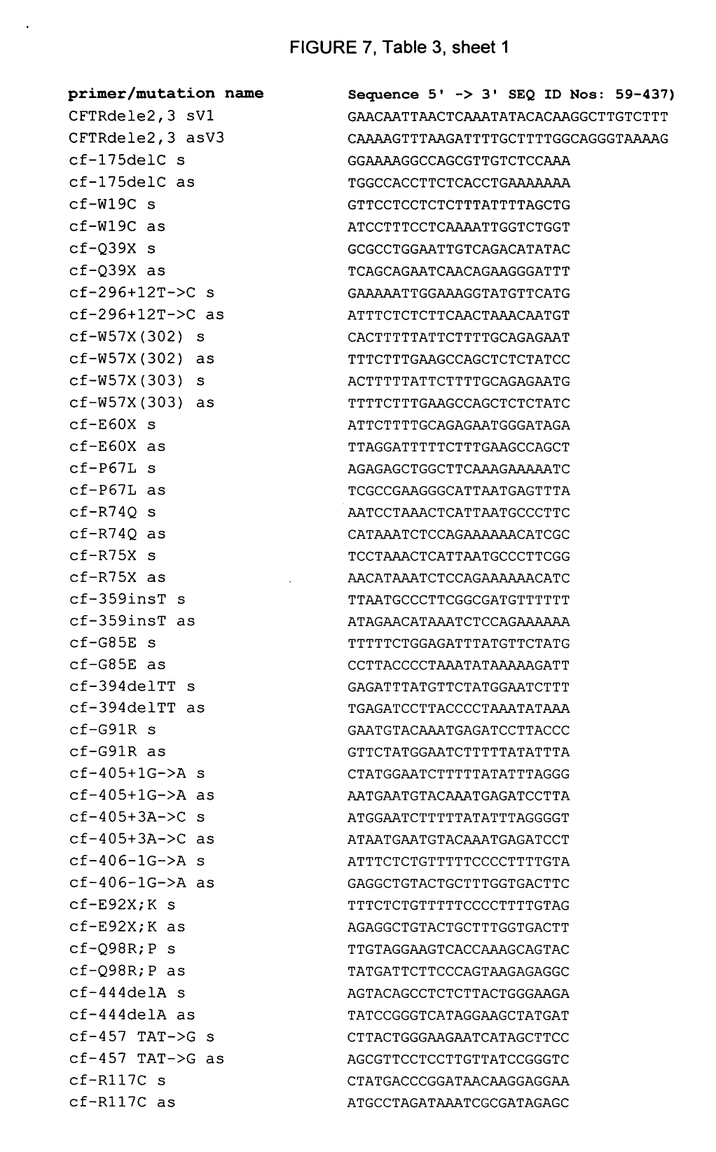

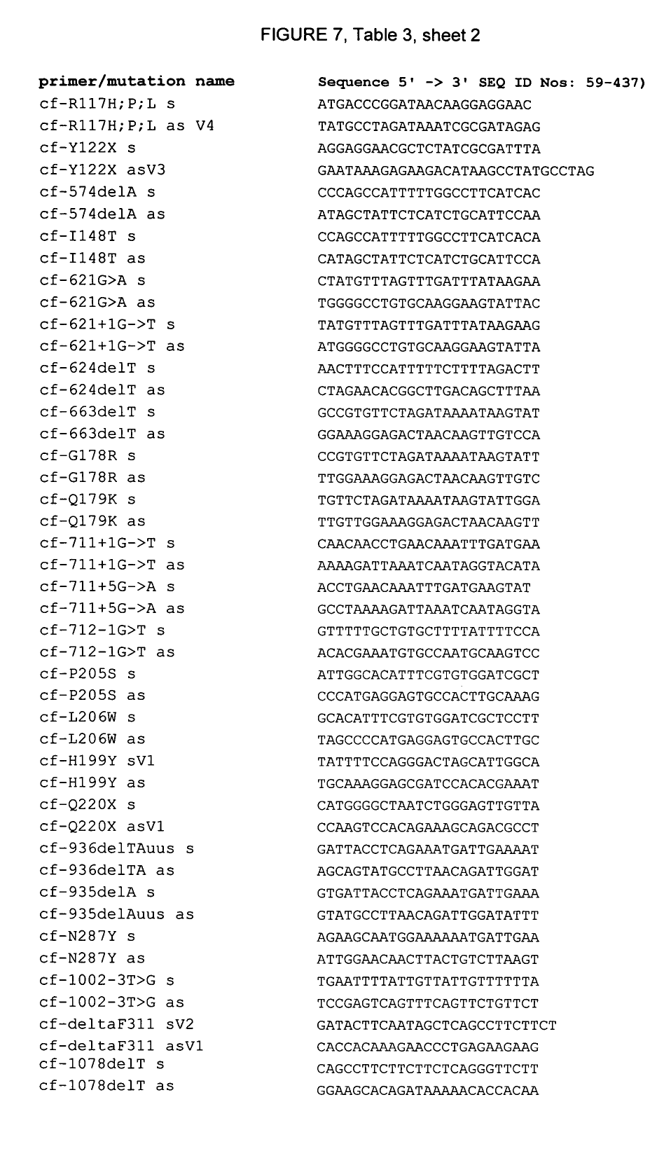

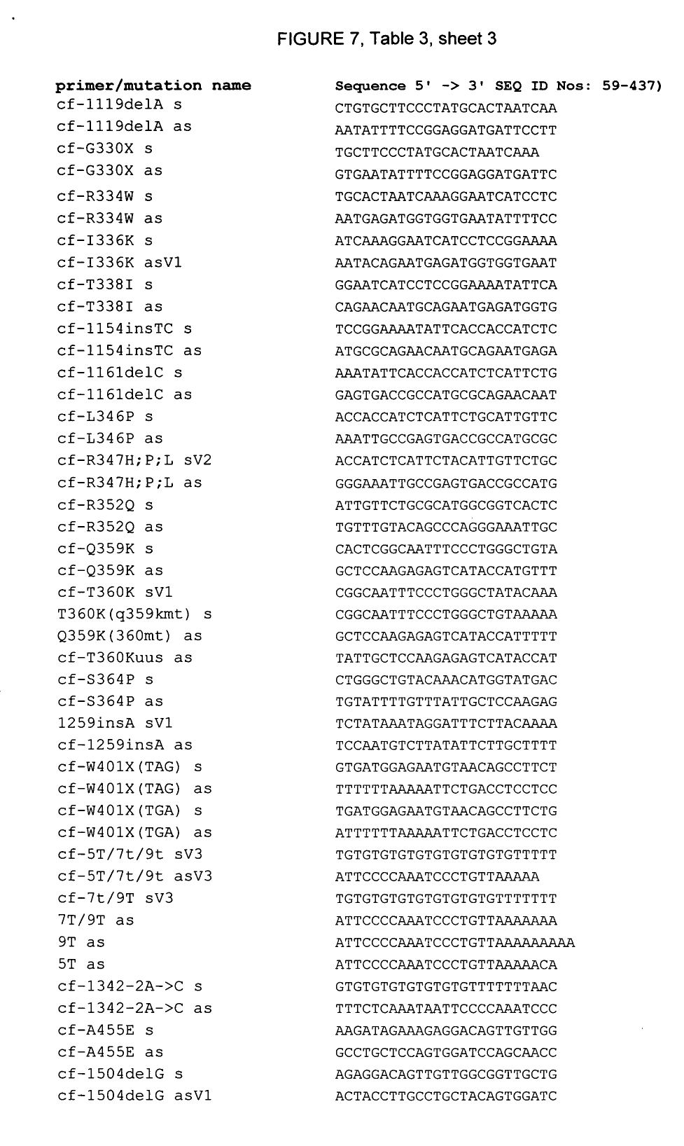

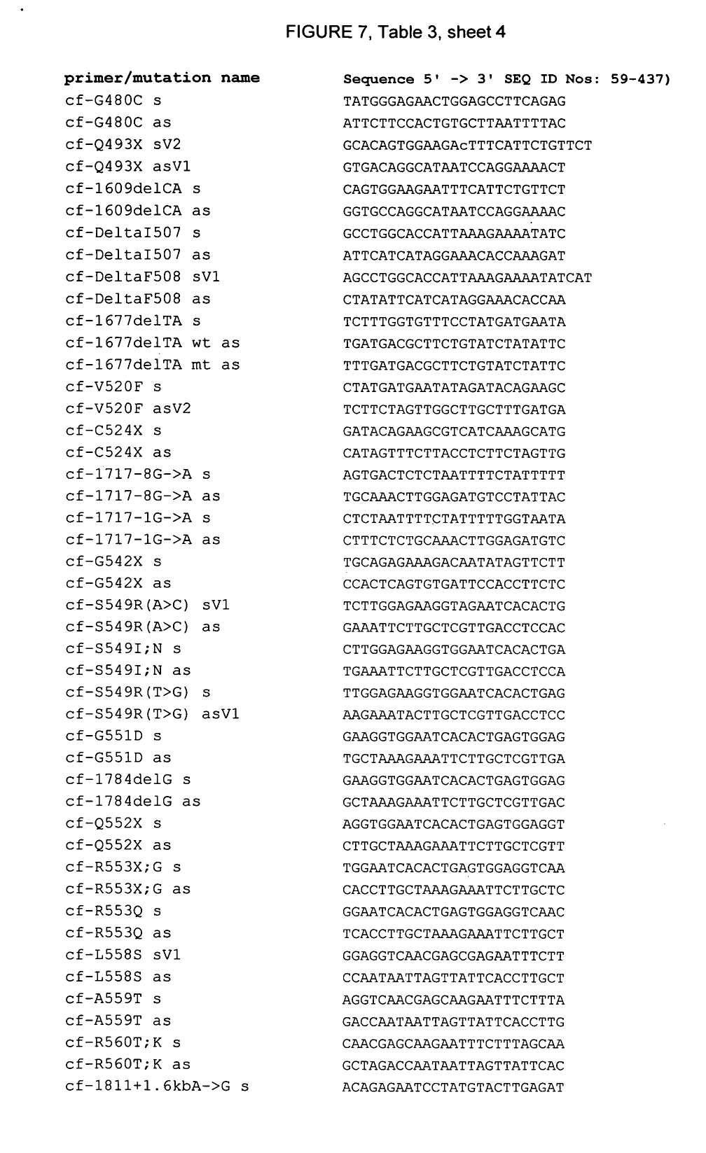

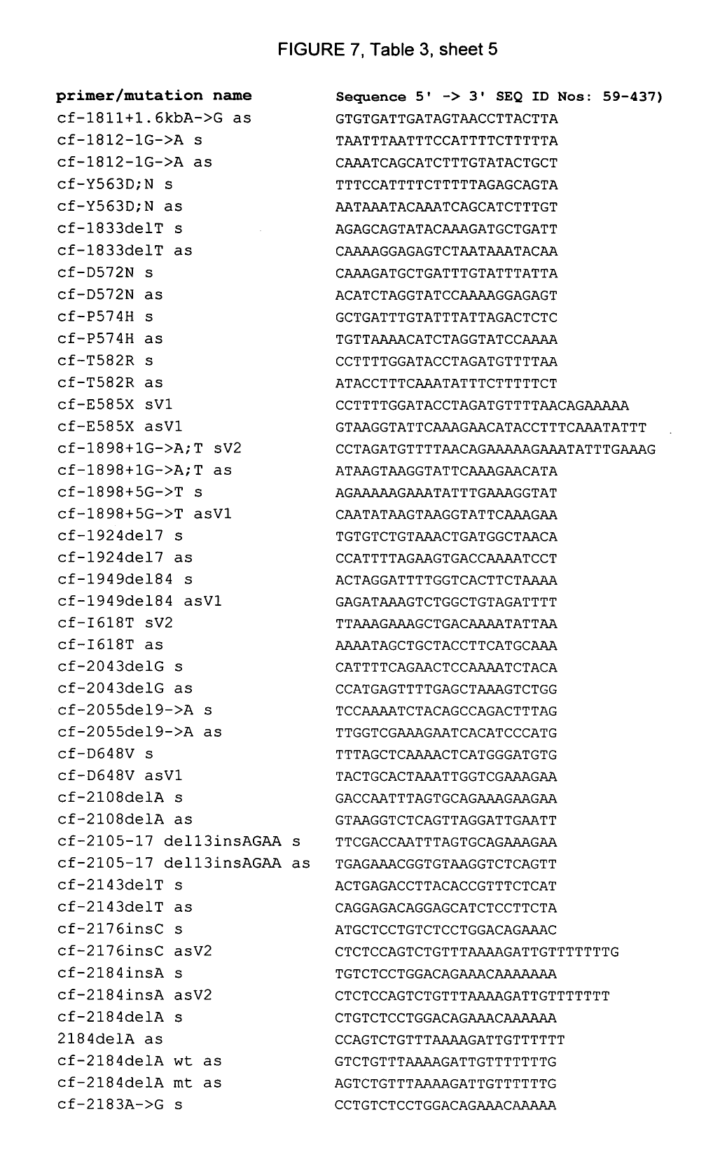

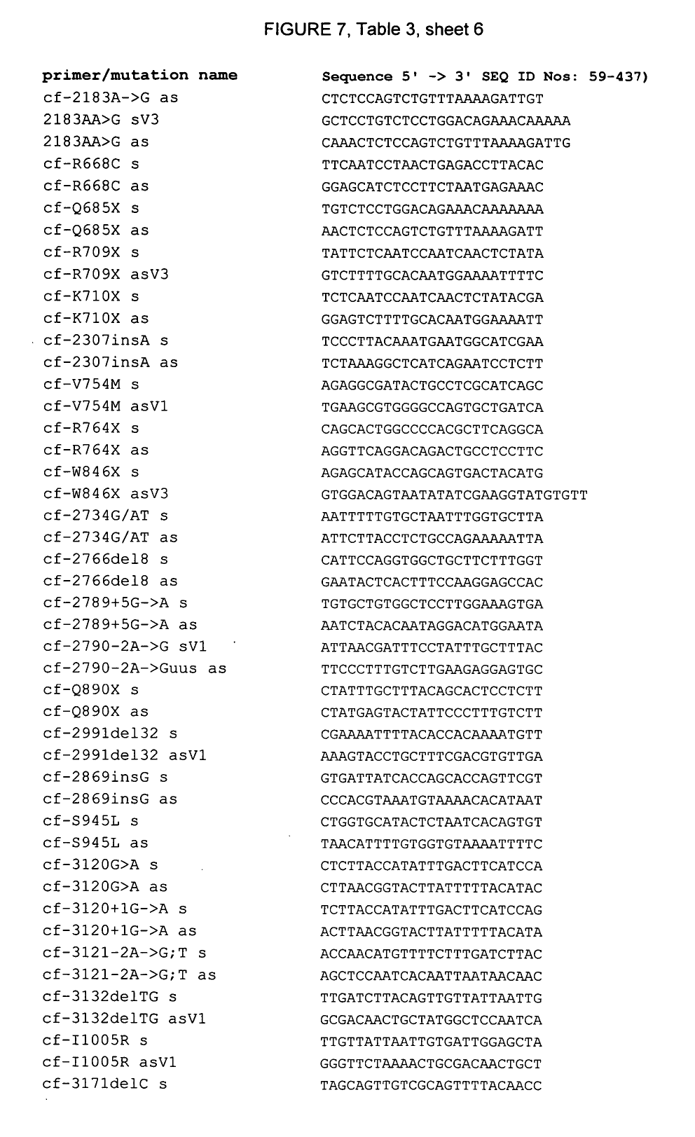

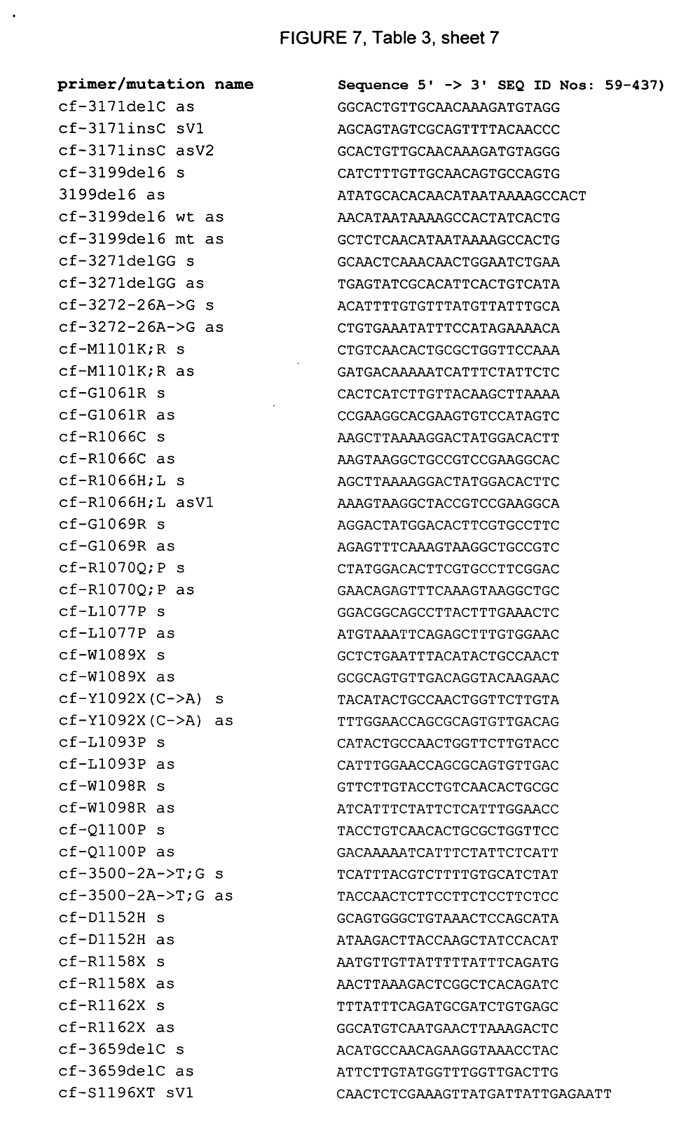

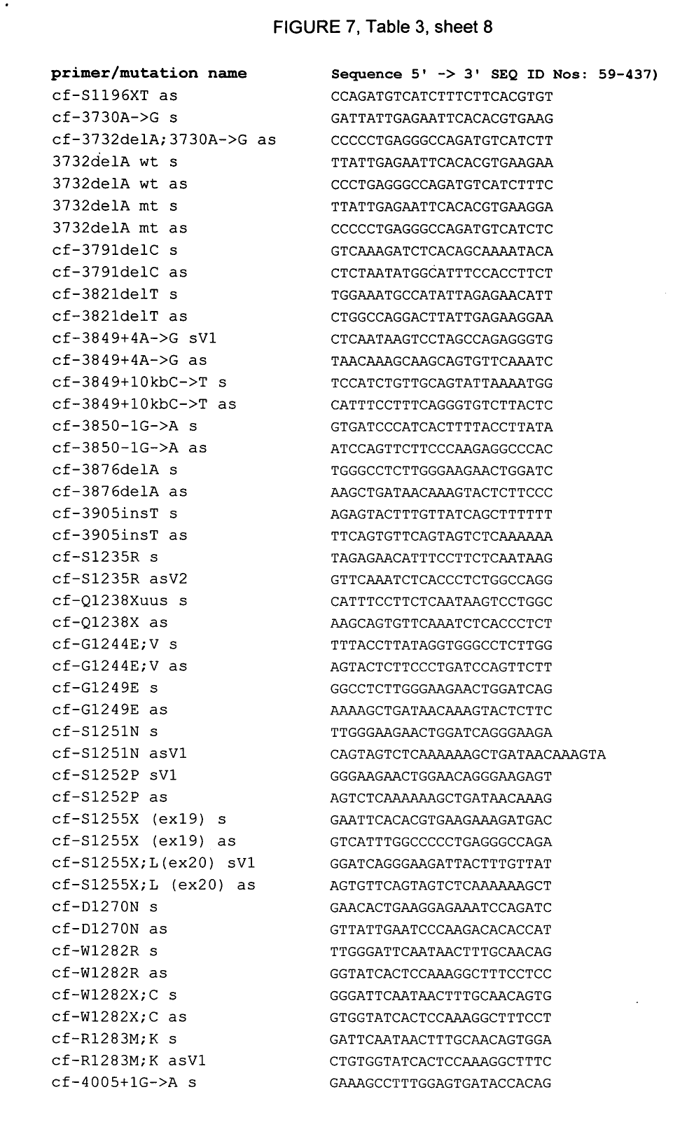

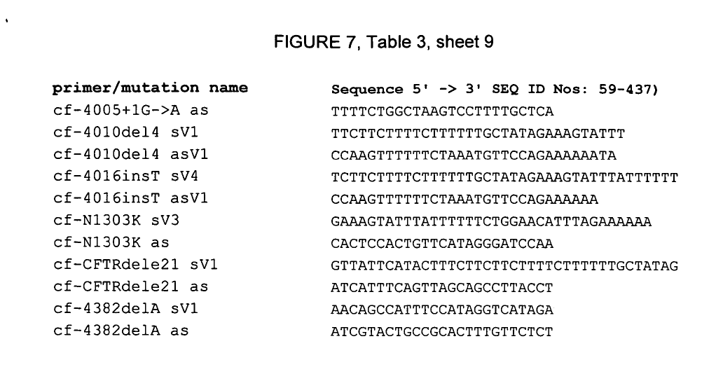

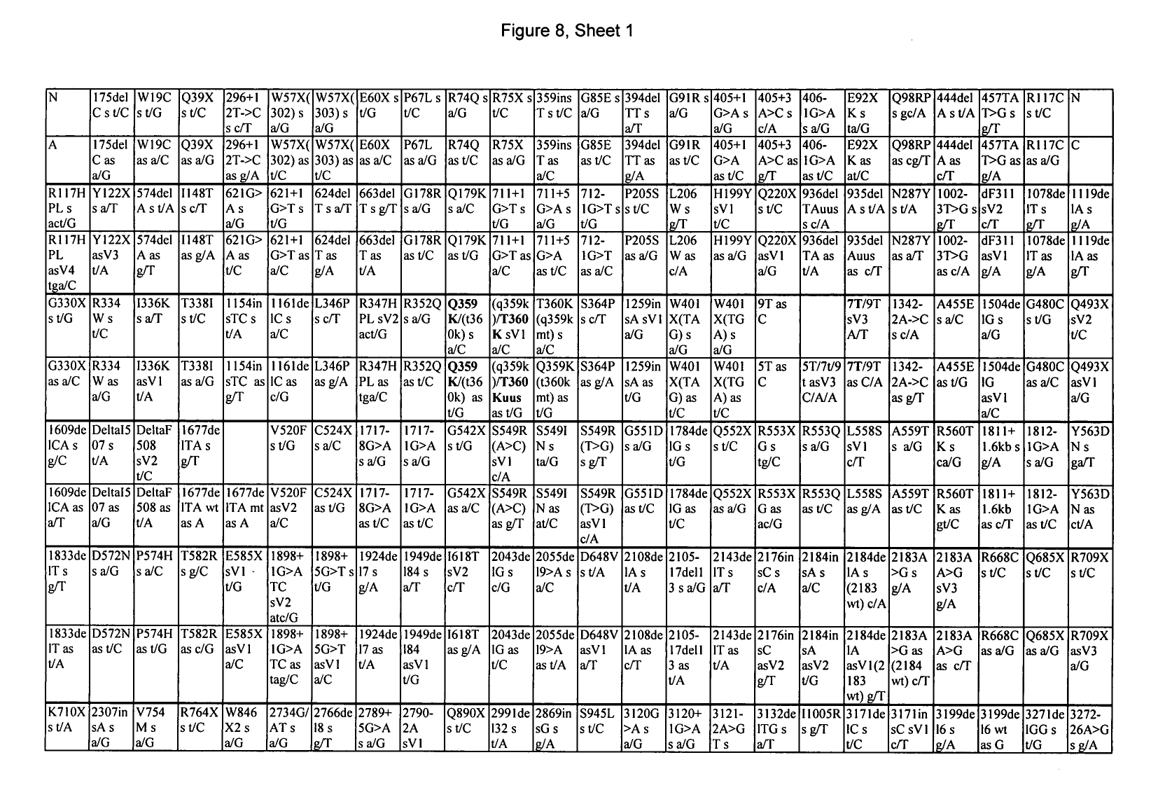

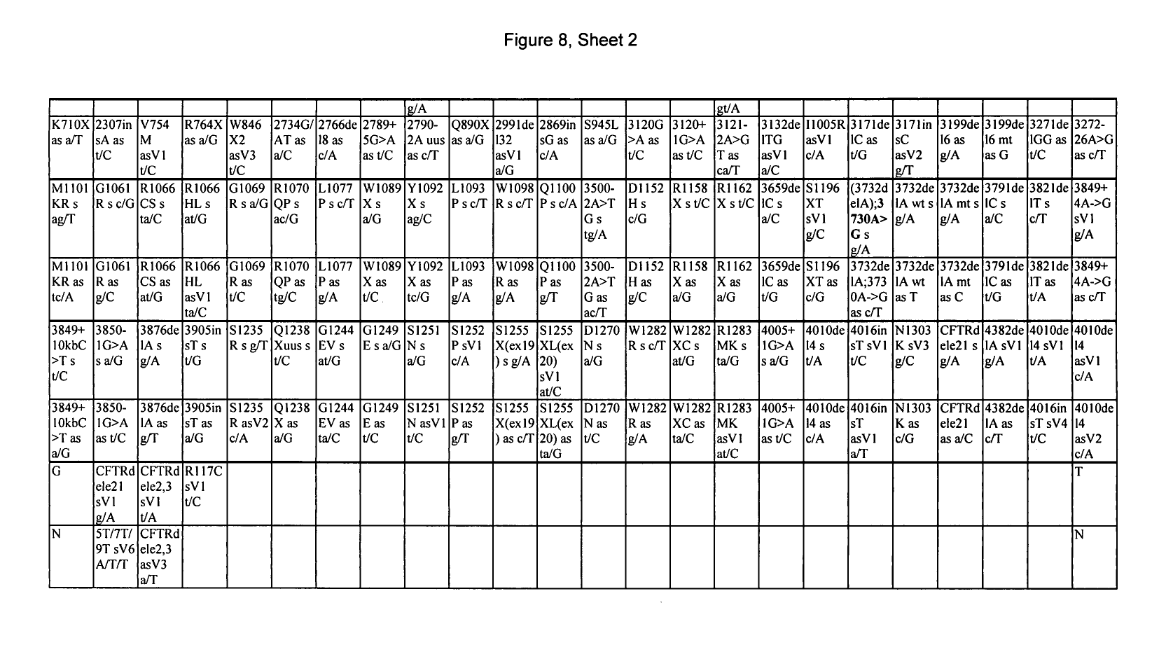

Each selected mutation in CFTR is identified by two unique 25-mer oligonucleotides, one for sense and one for antisense strand, though for some mutations three oligonucleotides are used (total of 379 oligonucleotides, Table 3, FIG. 7) as described in methods. These probes are annealed to the microarray slide in the grid pattern represented in Table 4, FIG. 8. Occasionally the oligonucleotides designed from the wild type CFTR sequence fail to perform the APEX reaction. The chief reason for APEX primer failure is the formation of self-annealing secondary structures that fail to hybridize or facilitate self-priming and extension. In order to obviate this problem, we designed new versions of the primers by incorporating a mismatch or a modified nucleotide at the 5′ or internal part of the primer. Such changes can reduce primer self-complementarity without compromising hybridization and primer extension

The redesigned primers are designated as V1 or higher in Table 3. In the case of secondary structures at the 3′ end, which is required for template annealing and extension, some versions with internal base substitutions can be attempted, but not all work. After final design of APEX primers, 182 mutations were detected in both the sense and antisense directions, 6 mutations from only the sense strand (antisense strand does not work reliably), and 13 mutations from only the antisense strand (sense strand does not work reliably.

APEX reactions were performed and detected with the Genorama Quattrolmager and analyzed with Genorama Genotyping Software 4.0, as described in Kurg et al., 2000. In general, the entire process, from PCR amplification (2 hr), PCR product purification (20 min), DNA fragmentation (1 hr), APEX reaction (15 min), visualization of results (6 min) and analysis of results (10-15 min) can be accomplished in 4 to 5 hours.

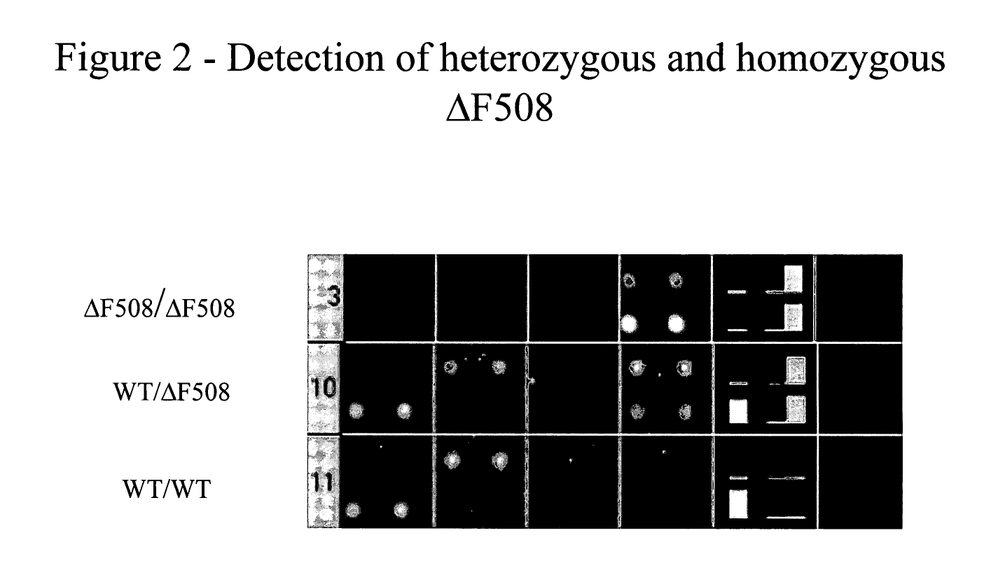

The results allow reliable and reproducible detection of wild type (WT) versus mutation sequence at each array position on the APEX CF microarray. Thus the assay is suitable both for screening of CF carriers (one heterozygote mutation in entire array) and for diagnosis of patients (two mutations, either heterozygous at two array sites or homozygous at one array site). Representative results are seen in FIGS. 1 and 2. FIG. 1 demonstrates results from three patient samples, one each of normal, heterozygous and homozygous, at the grid position for mutation 2183AA>G, a mutation in exon 13 (R domain) which has a 3.2% frequency in a screened sample of Italian patients (see e.g., the website having a url made up by placing “http://www.” before: “genet.sickkids.on.ca/cftr-cgi-bin/FullTable”. FIG. 2 shows the results from 3 patient samples at the grid position for the common mutation ΔF508, again one each for normal, heterozygous and homozygous at that position. In each case, the results accurately detect the sequence of both alleles for each patient sample.

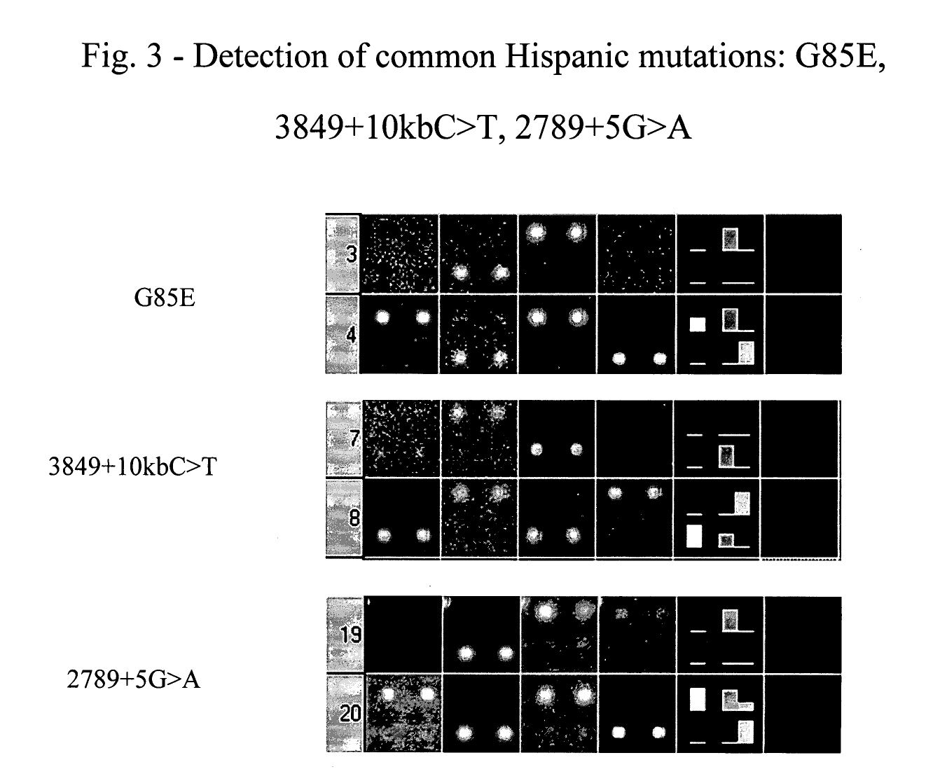

FIG. 3 shows the results of normal and patient samples at each of three grid sites for the mutations G85E, 3849+10 kbC>T, and 2789+5G>A. These mutations are common in the Hispanic population, which represents x% of the California population and which has a carrier frequency of 1:40. None of these mutations are on the currently recommended CF panel of mutations that are now commercially tested, but accurate screening for mutations such as these is essential in the ethnically diverse US population. In each case, the patient sample shows the presence of the mutation on one allele when compared to normal DNA samples.

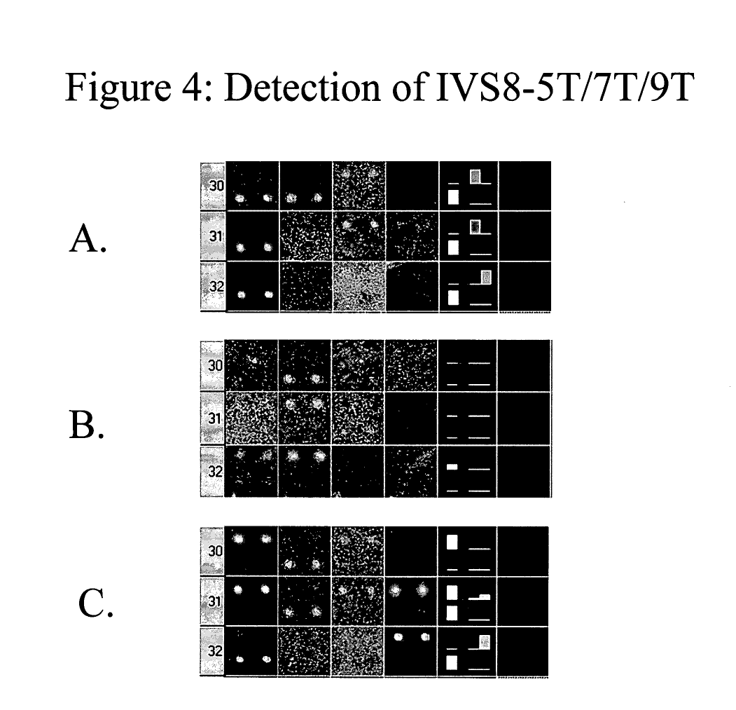

The CBAVD 5T/7T/9T mutation is the most technically difficult to detect and requires three sets of APEX primers for accurate detection. Representative results for three patient samples (5T/7T, 7T/9T, and 9T/9T) are shown in FIG. 4.

The CF APEX microarray was validated by means of 50 patient samples with different CFTR mutations. Mutation sites in which relevant patient samples could not readily be obtained were tested by means of 136 synthetic primers, designed as 50-mer oligonucleotides based on the wild type sequence but incorporating the mutation to be identified. Four sites were tested with patient samples and synthetic template DNA with comparable results. With this validation series, sensitivity (TP/TP+TN) was 99.9%, with 1 false negative signal (R11C/ΔF508 at position ΔF508). Specificity (TN/TN +FP) was 100%. The APEX reactions are reproducible.

It is evident that subject invention provides for a number of advantages as compared to existing CFTR gene mutation detection protocols. The microarray format of the described invention, combined with the simple and easy APEX technology procedure and low cost capital equipment for analysis, make the present methods more affordable than currently marketed versions of CF mutation detection assays. Furthermore, given the prevalence of the varied racial and ethnic groups in California alone, as well as the frequent inter-racial and inter-ethnic marriage, it is clear that a low cost, specific and sensitive assays detecting a greater number of mutations are needed, which assays are provided by the subject invention. The subject invention enables high-throughput testing at low cost on an individual basis and allows flexibility for future addition of mutations. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of determining whether a subject carries a Cystic Fibrosis Transmembrane Conductance Regulator (CFTR) gene mutation, said method comprising:
(a) contacting an array comprising a plurality of distinct nucleic acid CFTR gene mutation probes immobilized on a surface of a solid support with a nucleic acid sample from said subject to produce a sample contacted array;
(b) contacting said sample contacted array with a polymerase and at least two different distinguishably labeled dideoxynucleotides under primer extension conditions; and
(c) detecting the presence of any resultant terminally labeled nucleic acids immobilized on said substrate surface to determine whether said subject carries a CFTR gene mutation.

2. The method according to claim 1, wherein said array comprises at least about 50 probes from Table 1.

3. The method according to claim 1, wherein said nucleic acid sample is an amplified genomic sample.

4. The method according to claim 3, wherein said amplified genomic sample is a fragmented amplified genomic sample.

5. The method according to claim 5, wherein said fragmented amplified genomic sample is an enzymatically fragmented sample.

6. The method according to claim 1, wherein said array comprises a plurality of pairs of CFTR gene mutation probes, wherein each pair comprises a sense strand probe and an antisense strand probe.

7. The method according to claim 1, wherein said sample contacted array is contacted with four different distinguishably labeled ddNTPs.

8. The method according to claim 7, wherein said four different distinguishably labeled ddNTPs are ddATP, ddTTP, ddGTP and ddCTP.

9. The method according to claim 1, wherein said at least two dideoxynucleotides are labeled with fluorescent labels.

10. The method according to claim 9, wherein said detecting step comprises scanning said surface for said at least two different fluorescent labels.

11. The method according to claim 10, wherein said surface is scanned for four different fluorescent labels.

12. The method according to claim 1, wherein said method is a method for determining whether said subject is heterozygous for a CFTR gene mutation.

13. The method according to claim 1, wherein said method is a method for determining whether said subject is homozygous for a CFTR gene mutation.

14. An array comprising a plurality of at least about 50 distinct nucleic acid CFTR gene mutation probes immobilized on a surface of a solid support.

15. The array according to claim 14, wherein said at least about 50 distinct probes are from Table 1.

16. The array according to claim 14, wherein said array comprises a plurality of pairs of CFTR gene mutation probes, wherein each pair comprises a sense strand probe and an antisense strand probe.

17. The array according to claim 14, wherein said array comprises at least about 100 distinct nucleic acid CFTR gene mutation probes.

18. The array according to claim 17, wherein said array comprises at least about 150 distinct nucleic acid CFTR gene mutation probes.

19. A method of determining whether a subject carries a Cystic Fibrosis Transmembrane Conductance Regulator (CFTR) gene mutation, said method comprising:
(a) contacting an array comprising a plurality of at least about 50 distinct nucleic acid CFTR gene mutation probes immobilized on a surface of a solid support with a nucleic acid sample of target nucleic acids from said subject to produce a sample contacted array;
(b) detecting the presence of any resultant target nucleic acids immobilized on said substrate surface to determine whether said subject carries a CFTR gene mutation.

20. A kit for use determining whether a subject carries a Cystic Fibrosis Transmembrane Conductance Regulator (CFTR) gene mutation, said kit comprising:
(a) an array comprising a plurality of at least about 50 distinct nucleic acid CFTR gene mutation probes immobilized on a surface of a solid support; and
(b) at least two different distinguishably labeled dideoxynucleotides (ddNTPs).

21. The kit according to claim 20, wherein said at least about 50 distinct probes are from Table 1.

22. The kit according to claim 20, wherein said array comprises a plurality of pairs of CFTR gene mutation probes, wherein each pair comprises a sense strand probe and an antisense strand probe.

23. The kit according to claim 20, wherein said array comprises at least about 100 distinct nucleic acid CFTR gene mutation probes.

24. The kit according to claim 23, wherein said array comprises at least about 150 distinct nucleic acid CFTR gene mutation probes.

25. The kit according to claim 20, wherein said sample contacted array is contacted with four different distinguishably labeled ddNTPs.

26. The kit according to claim 20, wherein said kit comprises four different distinguishably labeled ddNTPs are ddATP, ddTTP, ddGTP and ddCTP.

27. The kit according to claim 20, wherein said at least two dideoxynucleotides are labeled with fluorescent labels.
